(19) United States
(12) Patent Application Publication
(10) Pub. No.: US 2023/0074252 A1
(43) Pub. Date: Mar. 9, 2023

(54) DOSAGES AND METHODS FOR TREATING PULMONARY ARTERIAL HYPERTENSION WITH RODATRISTAT

(71) Applicant: Altavant Sciences GmbH, Basel (CH)

(72) Inventors: Stephen A. Wring, South Boston, VA (US); Michelle Palacios, Garner, NC (US); Katelyn Reighard Crizer, Chapel Hill, NC (US); Bill Symonds, Cary, NC (US); Daniel Patterson, Durham, NC (US)

(21) Appl. No.: 17/759,700

(22) PCT Filed: Jan. 29, 2021
(86) PCT No.: PCT/IB2021/000046

§ 371 Date: Jul. 28, 2022

Related U.S. Application Data
(60) Provisional application No. 62/968,651, filed on Jan. 31, 2020.

(51) Int. Cl. A61K 31/506; A61K 31/4985

(52) U.S. Cl. CPC A61K 31/506; A61K 31/4985

(57) ABSTRACT

There is a daily dosage for treating pulmonary arterial hypertension (PAH) of two discrete dosage forms each having up to 600 mg of rodatristat ethyl or up to 1200 mg of rodatristat ethyl once per day. There are methods for treating PAH in human patients in need thereof via administration of the dosage forms. There is also a method for treating PAH via daily administration of an amount of a first compound of rodatristat ethyl and an amount of a second compound selected from among ambrisentan, sildenafil, tadalafil, bosentan, treprostinil, selexipag, macitentan, and a combination of two or more thereof. The combination results in an additive or synergistic reduction in symptoms or side effects of PAH compared to the two alone with a low risk for drug-drug interaction, such with a C_{max, unbound} in the bloodstream of less than 0.028 μM. The combination can also result in neither rodatristat ethyl nor its active metabolite rodatristat substantially impacting metabolism of the second compound in the bloodstream. The combination can also result in rodatristat ethyl and its active metabolite rodatristat exhibiting an IC₅₀ of ≥30 μM for the enzyme group consisting of CYP1A2, CYP2C9, CYP2C19, and CYP2D6 in the bloodstream. The combination can also result in rodatristat ethyl and its active metabolite rodatristat exhibiting an IC₅₀ of ≥30 μM for the enzyme group consisting of CYP1A2, CYP2B6, CYP2C9, CYP2C19, CYP2D6, and CYP3A4.

### BACKGROUND OF THE DISCLOSURE

### 1. Field of the Disclosure

The present disclosure relates to daily dosages for treating pulmonary arterial hypertension with medicinal actives. The present disclosure further relates to methods for treating pulmonary arterial hypertension with medicinal actives.

### 2. Description of the Related Art

Pulmonary arterial hypertension (PAH) is a severe, incurable disease characterized by remodeling of the pulmonary arterial bed, leading to elevations in resting mean pulmonary artery pressures, subsequent right ventricular hypertrophy, and eventually, right heart failure and death. Am Heart J. 2011 August; 162(2): 201-13, doi: 10.1016/j.ahj.2011.05.012. Epub 2011 Jul. 13. Current standard-of care therapies for PAH target three different pathways—namely, the endothelin-1 pathway, the nitric oxide pathway, and the prostacyclin pathway. Humbert M. Ghofrani H-A. Thorax 2016: 71; 73-83. doi: 10.1136/thoraxjnl-2015-207170.

Patients with PAH have a deficiency of prostacyclin and prostacyclin synthase. Selexipag was the first orally active, selective, long-acting, nonprostanoid prostacyclin receptor agonist approved for the treatment of PAH [Scott, 2016]. Selexipag is hydrolyzed to its active metabolite ACT-333679, which is approximately 37-fold more potent than selexipag [Kaufmann, 2015, Bruderer et al, 2017]. In vitro experiments have shown that selexipag and ACT-333679 undergo oxidative metabolism by the cytochrome P450 (CYP) enzymes, CYP2C8 and CYP3A4. A drug-drug interaction study conducted with gemfibrozil (a strong irreversible inhibitor of CYP2C8) and selexipag determined that concomitant administration with selexipag and strong inhibitors of CYP2C8 must be avoided based on an 11-fold increase in ACT-333679 AUC_{0-∞}. The incidence and/or intensity of AEs reported after concomitant administration of selexipag and gemfibrozil were significantly higher than those following the administration of selexipag alone, which is in line with the observed increase in exposure to the active metabolite ACT-333679, the major contributor to the effect of selexipag in humans. [Bruderer et al., 2017].

Rodatristat ethyl (RVT-1201) is a pro-drug for the active tryptophan hydroxylase (TPH1) inhibitor rodatristat (KAR5417) and is in development for PAH. Following oral administration, rodatristat ethyl is rapidly absorbed and hydrolyzed to KAR5417. It would be desirable to have a dosage system and method of combining rodatristat ethyl or KAR5417 with an FDA-approved PAH pharmaceutical active to provide enhanced efficacy in treating the symptoms or side effects of PAH compared to either the rodatristat ethyl/KAR5417 or the FDA-approved active alone. It would further be desirable to have a dosage system and method that provides a high, steady state exposure by Day 7 in a human patient receiving doses of up to 600 mg BID rodatristat ethyl.

### SUMMARY OF THE DISCLOSURE

According to the present disclosure, there is provided a daily dosage for treating pulmonary arterial hypertension. The daily dosage has two discrete dosage forms, wherein each of the dosage forms includes an amount of up to 600 mg of rodatristat ethyl.

Further according to the present disclosure, there is provided a method for treating pulmonary arterial hypertension. The method has the step of administering to a human patient in need thereof an individual dosage amount of up to 600 mg of rodatristat ethyl twice per day.

Further according to the present disclosure, there is provided another method for treating pulmonary arterial hypertension that includes the step of administering to a human patient in need thereof an amount of up to 1200 mg of rodatristat ethyl once per day.

Further according to the present disclosure, there is provided another method for treating pulmonary arterial hypertension. The method has the step of administering daily to a human patient in need thereof (A) an amount of up to 1200 mg of a first compound of rodatristat ethyl and (B) an amount of a second compound selected from the group consisting of ambrisentan, sildenafil, tadalafil, bosentan, treprostinil, selexipag, and two or more of the foregoing. The first compound and the second compound are in a combination that results in an additive or synergistic reduction in symptoms of pulmonary arterial hypertension compared to the first compound and the second compound alone with a low risk for drug-drug interactions.

Further according to the present disclosure, there is provided another method for treating pulmonary arterial hypertension that includes a step of administering to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing. The first compound and the second compound are in a combination that results in a synergistic or additive reduction in symptoms of pulmonary arterial hypertension compared to the first compound and the second compound alone, and wherein the amount of rodatristat ethyl results in a C_{max, unbound} in the bloodstream of less than 0.028 μM.

Further according to the present disclosure, there is provided another method for treating pulmonary arterial hypertension that includes a step of administering to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing, wherein the first compound and the second compound are in a combination that results in a synergistic reduction in side effects of pulmonary arterial hypertension compared to the first compound and the second compound alone or additively, and wherein the amount of KAR5417 results in a C_{max, unbound} in the bloodstream of less than 0.028 μM.

Further according to the present disclosure, there is provided another method for treating pulmonary arterial hypertension that includes a step of administering to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing, wherein the first compound and the second compound are in a combination that results in a synergistic reduction in side effects of pulmonary arterial hypertension compared to the first compound and the second compound alone or additively, and wherein the sum amount of rodatristat ethyl and KAR5417 results in a combined C_{max, unbound} in the bloodstream of less than 0.028 μM.

Further according to the present disclosure, there is provided yet another method for treating pulmonary arterial hypertension that includes a step of administering daily to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan and two or more of the foregoing. The first compound and the second compound are in a combination that results in a synergistic reduction in side effects of pulmonary arterial hypertension compared to the first compound and the second compound alone or additively, and wherein neither the first compound nor its active metabolite rodatristat substantially impact metabolism of the second compound in the bloodstream.

Further according to the present invention, there is provided yet another method for treating pulmonary arterial hypertension that includes a step of administering daily to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan and two or more of the foregoing. The rodatristat ethyl and its active metabolite rodatristat exhibit an IC50 of ≥30 μM for one or more of the enzyme group consisting of the enzyme group consisting of CYP1A2, CYP2C9, CYP2C19, and CYP2D6 in the bloodstream.

Further according to the present invention, there is provided yet another method for treating pulmonary arterial hypertension that includes a step of administering daily to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan and two or more of the foregoing. The rodatristat ethyl and its active metabolite rodatristat exhibit an IC50 of ≥30 μM for one or more of the enzyme group consisting of the enzyme group consisting of CYP1A2, CYP2B6, CYP2C9, CYP2C19, CYP2D6, and CYP3A4.

Further according to the present disclosure, there is provided a method for treating pulmonary arterial hypertension that includes the step of administering to a human patient in need thereof a therapeutically effect amount of rodatristat ethyl, Form 3.

### BRIEF DESCRIPTION OF THE DRAWINGS

Embodiments of the present disclosure are described herein with reference to the following figures.

FIG. 1 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate according to the present disclosure (crystalline Form 3).

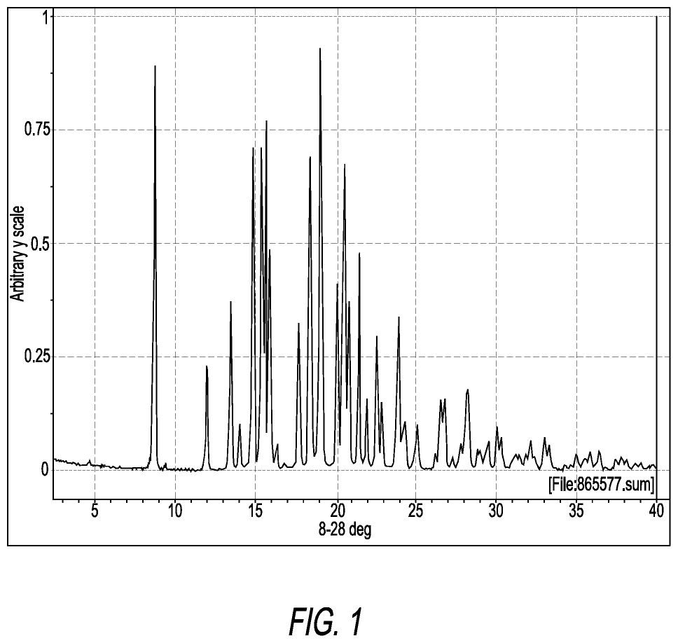

FIG. 2 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate of a different polymorphic form than that of FIG. 1 (crystalline Form 1).

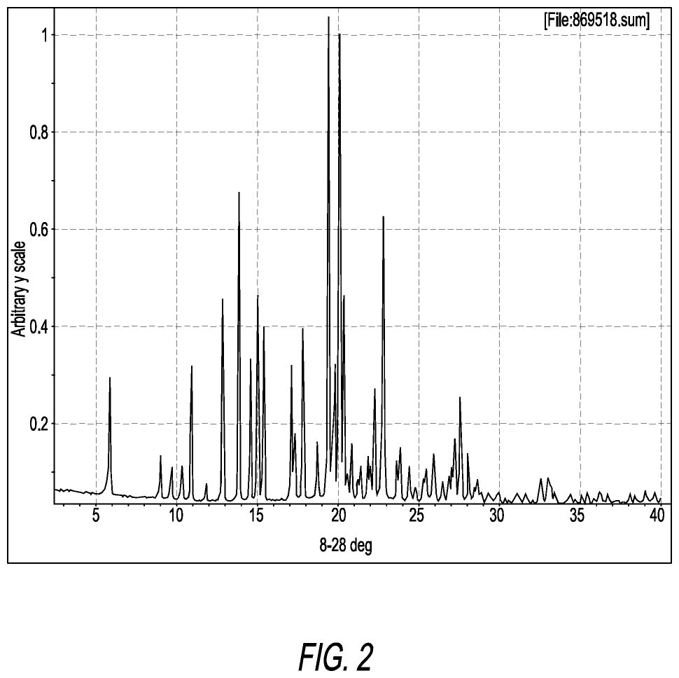

FIG. 3 shows the predicted Cₘₐₓ for the 600 mg BID dose for RE with a low risk for CYP inhibition.

FIG. 4 shows the predicted Cₘₐₓ for the 600 mg BID dose for R predicts with a low risk for CYP inhibition.

FIG. 5 shows a indexing solution for RVT-1201-KAR5585, Lot CS14-075Aa-1704, Form 3, LIMS No. 470337.

### DETAILED DESCRIPTION OF THE DISCLOSURE

Rodatristat is metabolically stable and has low potential for drug interactions with PAH medications. There is low potential for metabolic drug-drug interactions (DDIs) between rodatristat ethyl and approved medications for pulmonary arterial hypertension (PAH).

Rodatristat ethyl (RE) is in Phase 2 clinical development (ELEVATE 2 Study) for PAH. RE is an orally bioavailable pro-drug for the tryptophan hydroxylase (TPH) inhibitor, rodatristat (R). TPH1 is the rate-limiting enzyme for peripheral biosynthesis of serotonin (5-HT) and is up-regulated in PAH. Excess 5-HT has been implicated in the pathology of PAH. See MacLean, M. R. 2018. The serotonin hypothesis in pulmonary hypertension revisited: targets for novel therapies. Pulm Circ. 8 (2). 1-9.

Rodatristat ethyl is a pro-drug for the active tryptophan hydroxylase (TPH1) inhibitor KAR5417 and is in development for PAH. Following oral administration, rodatristat ethyl is rapidly absorbed and hydrolyzed to KAR5417. The highest exposures achieved in healthy subjects to date were reached on Day 7 following 800 mg twice per day (BID) doses of rodatristat ethyl. Rodatristat ethyl and KAR5417 inhibit CYP2C8 in vitro, but have been determined to have a weak potential to cause a clinically relevant inhibitory interaction with this enzyme in humans receiving doses of up to 600 mg BID rodatristat ethyl.

PAH treatment sometimes employs combination therapy regimens and understanding drug-drug interactions (DDI) potential is an important feature in managing patient care. A drug-drug interaction study has been conducted to determine the effect, if any, of rodatristat ethyl steady-state dosing has on the pharmacokinetics (PK) of selexipag and its metabolite ACT-333679.

### PAH Pharmaceutical Actives

(S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate is referred to herein rodatristat ethyl (RE) and in the literature also as KAR5585 and RVT-1201. Rodatristat ethyl has the following structure:

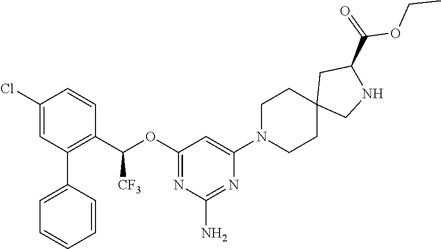 [SMILES: CCOC(=O)[C@@H]1CC2(CCN(c3cc(O[C@H](c4ccc(Cl)cc4-c4ccccc4)C(F)(F)F)nc(N)n3)CC2)CN1]

The amorphous form of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate can be prepared by the method set forth in Example 63i of U.S. Pat. No. 9,199,994, which is incorporated herein by reference in its entirety. The amorphous form can then be converted to crystalline form by methods described in U.S. Patent Publication 2020/0148681 A1, published May 14, 2020, which is incorporated herein by reference in its entirety. Forms 1 and 3 can be prepared by the methods set forth in U.S. Patent Publication 2020/0148681 A1.

Form 3 exhibits the following x-ray powder diffraction pattern (XRPD):

| Peak position (°2θ) | d space (Å) | Intensity (%) |
|---|---|---|
| 8.78 ± 0.20 | 10.077 ± 0.235 | 90 |
| 12.00 ± 0.20 | 7.375 ± 0.125 | 25 |
| 13.47 ± 0.20 | 6.573 ± 0.099 | 39 |
| 14.02 ± 0.20 | 6.316 ± 0.091 | 12 |
| 14.87 ± 0.20 | 5.956 ± 0.081 | 71 |
| 15.39 ± 0.20 | 5.757 ± 0.075 | 72 |
| 15.61 ± 0.20 | 5.677 ± 0.073 | 78 |
| 15.89 ± 0.20 | 5.576 ± 0.071 | 50 |
| 16.31 ± 0.20 | 5.434 ± 0.067 | 7 |
| 17.70 ± 0.20 | 5.011 ± 0.057 | 34 |
| 18.45 ± 0.20 | 4.809 ± 0.052 | 70 |
| 19.05 ± 0.20 | 4.658 ± 0.049 | 100 |
| 20.12 ± 0.20 | 4.413 ± 0.044 | 42 |
| 20.57 ± 0.20 | 4.317 ± 0.042 | 68 |
| 20.84 ± 0.20 | 4.262 ± 0.041 | 39 |
| 21.46 ± 0.20 | 4.141 ± 0.039 | 49 |
| 21.94 ± 0.20 | 4.051 ± 0.037 | 18 |
| 22.56 ± 0.20 | 3.941 ± 0.035 | 31 |
| 22.90 ± 0.20 | 3.884 ± 0.034 | 17 |
| 23.90 ± 0.20 | 3.723 ± 0.031 | 35 |
| 24.32 ± 0.20 | 3.660 ± 0.030 | 13 |
| 25.07 ± 0.20 | 3.552 ± 0.028 | 12 |
| 26.54 ± 0.20 | 3.359 ± 0.025 | 17 |
| 26.76 ± 0.20 | 3.332 ± 0.025 | 18 |
| 27.79 ± 0.20 | 3.210 ± 0.023 | 8 |
| 28.21 ± 0.20 | 3.163 ± 0.022 | 19 |
| 29.48 ± 0.20 | 3.030 ± 0.020 | 9 |

The x-ray powder diffraction pattern is carried out with a Cu Kα radiation source according to the following method:

### PANalytical X'Pert PRO MPD Diffractometer—Reflection Geometry

Selected XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for each pattern are displayed above the image in the Data section of this report including the divergence slit (DS) and the incident-beam SS.

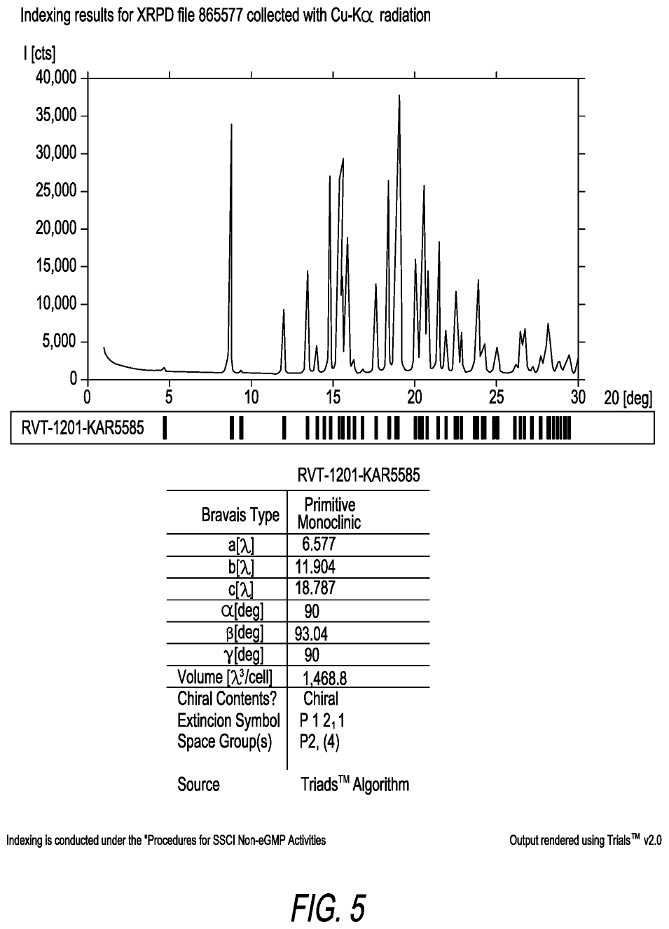

Figures were generated using validated software PatternMatch v 2.3.6. An indexing solution for rodatristat ethyl is shown in FIG. 5.

The metabolite of rodatristat ethyl is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid, which is of the formula

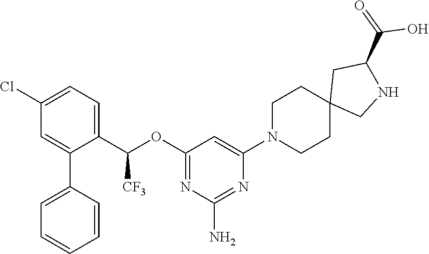 [SMILES: Nc1nc(O[C@H](c2ccc(Cl)cc2-c2ccccc2)C(F)(F)F)cc(N2CCC3(CC2)CN[C@H](C(=O)O)C3)n1]

(S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid is referred to herein as rodatristat. Rodatristat is also referred to in the literature as KAR5417. When rodatristat ethyl enters the bloodstream, it substantially converts to rodatristat. The amorphous form of KAR5417 can be prepared by the method set forth in Example 34c of U.S. Pat. No. 9,199,994, which again is incorporated herein in its entirety.

### FDA-Approved PAH Pharmaceutical Actives

The following FDA-approved pharmaceutical actives are useful in combination with rodatristat and rodatristat ethyl in treating PAH.

Sildenafil citrate is the citrate salt of sildenafil, a selective inhibitor of cyclic guanosine monophosphate (cGMP)-specific phosphodiesterase type 5 (PDES). Sildenafil citrate is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate and has the following structural formula:

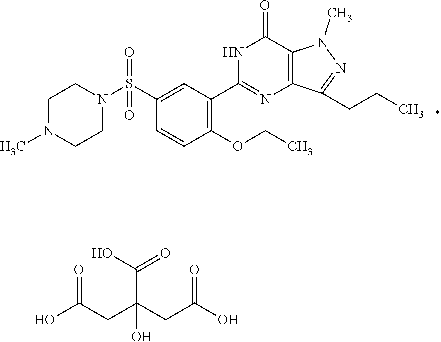 [SMILES: CCCc1nn(C)c2c(=O)[nH]c(-c3cc(S(=O)(=O)N4CCN(C)CC4)ccc3OCC)nc12 ; O=C(O)CC(O)(CC(=O)O)C(=O)O]

Tadalafil, a PDE-5 inhibitor, is designated chemically as (2R,8R)-2-(2H-1,3-benzodioxol-5-yl)-6-methyl-3,6,17-triazatetracyclo[8.7.0.0³,⁸.0¹¹,¹⁶]heptadeca-1(10),11,13,15-tetraene-4,7-dione and has the following structural formula:

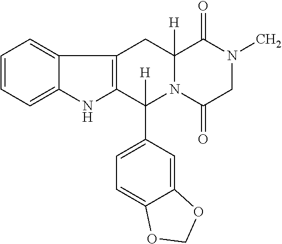

Bosentan is an endothelin receptor antagonist belonging to a class of highly substituted pyrimidine derivatives, with no chiral centers. It is designated chemically as 4-tert-butyl-N-[6-(2-hydroxy-ethoxy)-5-(2-methoxy-phenoxy)-[2,2′]-bipyrimidin4-yl]-benzenesulfonamide monohydrate and has the following structural formula:

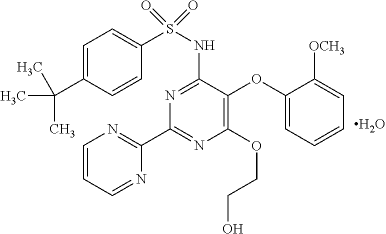

Ambrisentan is an endothelin receptor antagonist selective for the endothelin type-A (ETA) receptor. The chemical name of ambrisentan is (+)-(2S)-2-[(4,6-dimethylpyrimidin yl)oxy]-3-methoxy-3,3-diphenylpropanoic acid. It contains a single chiral center determined to be the (S) configuration and has the following structural formula:

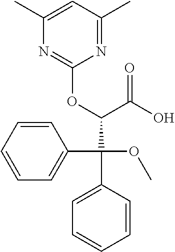 [SMILES: COC(c1ccccc1)(c1ccccc1)[C@H](Oc1nc(C)cc(C)n1)C(=O)O]

Selexipag is prostacyclin receptor agonist that causes vasodilation in pulmonary vasculature. Selexipag is class of pyrazines that is N-(methanesulfonyl)-2-{4-[(propan-2-yl)(pyrazin-2-yl)amino]butoxy}acetamide carrying two additional phenyl substituents at positions 5 and 6 on the pyrazine ring. It has the following structural formula:

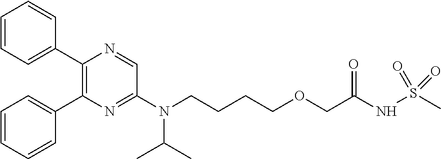 [SMILES: CC(C)N(CCCCOCC(=O)NS(C)(=O)=O)c1cnc(-c2ccccc2)c(-c2ccccc2)n1]

Treprostinil is a prostacycline vasodilator. Treprostinil (sodium) is (1R,2R,3aS,9aS)-[[2,3,3a,4,9,9a-Hexahydro-2-hydroxy-1-[(3S)-3-hydroxyoctyl]-1Hbenz[f]inden-5-yl]oxy]acetic acid monosodium salt. It has the following structural formula:

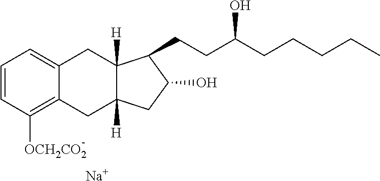

Macitentan is an endothelin receptor antagonist (ERA). Macitentan blocks the ET1-dependent rise in intracellular calcium by inhibiting the binding of ET-1 to ET receptors. Blocking of the ETA receptor subtype seems to be of more importance in the treatment of PAH than blocking of ETB, likely because there are higher numbers of ETA receptors than ETB receptors in pulmonary arterial smooth muscle cells. The chemical formula is N-[5-(4-Bromophenyl)-6-[2-[(5-bromo-2-pyrimidinyl)oxy]ethoxy]-4-pyrimidinyl]-N′-propyl-sulfamide. The CAS number is 441798-33-0. The structural formula is the following:

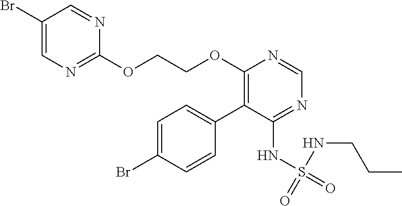 [SMILES: CCCNS(=O)(=O)Nc1ncnc(OCCOc2ncc(Br)cn2)c1-c1ccc(Br)cc1]

As used herein, the phrase “therapeutically effective amount” refers to the amount of active compound or pharmaceutical agent that elicits the biological or medicinal response in a tissue, system, individual or human that is being sought by a researcher, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

A compound of this disclosure can be administered orally, subcutaneously, topically, parenterally, by inhalation spray or rectally in dosage unit formulations containing conventional non-toxic pharmaceutically acceptable carriers, adjuvants and vehicles. Parenteral administration can involve subcutaneous injections, intravenous or intramuscular injections or infusion techniques.

In some embodiments, pharmaceutical compositions can be prepared as solid dosage forms for oral administration (e.g., capsules, tablets, pills, dragees, powders, granules and the like). A tablet can be prepared by compression or molding. Compressed tablets can include one or more binders, lubricants, glidants, inert diluents, preservatives, disintegrants, or dispersing agents. Tablets and other solid dosage forms, such as capsules, pills and granules, can include coatings, such as enteric coatings.

The amount of rodatristat ethyl and rodatristat to be administered will vary depending on factors such as the following: the compound selected, method of administration, release profile, and composition formulation. Typically, for rodatristat ethyl or rodatristat in an oral dosage form to treat or prevent a disease, particularly PH/PAH/APAH/IPAH/FPAH, a typical dosage will be a therapeutic amount of about 1 mg/kg/day to about 50 mg/kg/day and more typically from about 5 mg/kg/day to about 30 mg/kg/day, based on the weight of the patient. A most preferred compound is rodatristat ethyl in crystalline Form 3. Individual oral dosage forms typically have therapeutic amounts from about 50 mg to about 3000 mg of rodatristat ethyl and additional amounts of one or more pharmaceutically acceptable excipients. Other useful individual oral dosage forms can, by way of example, have rodatristat ethyl or rodatristat in amounts of 100 mg, 150 mg, 200 mg, 250 mg, 300 mg, 350 mg, or 400 mg, 450 mg, 500 mg, 550 mg, 575 mg, 600 mg, 625 mg, 650 mg, 675 mg, 700 mg, 725 mg, 750 mg, 775 mg, 800 mg, 900 mg, 950 mg, 1000 mg, 1050 mg, 1100 mg, 1150 mg, and about 1200 mg, particularly 600 mg and 1200 mg. A preferred dosage is 1200 mg. Other amounts between 50 mg to 3000 mg are possible, for example, from about 325 mg to about 475 mg, from about 350 mg to about 500 mg, from about 375 to about 525 mg, from about 400 mg to about 550 mg, from about 425 mg to about 575 mg, from about 450 mg to about 600 mg, from about 475 mg to about 625 mg, from about 500 mg to about 650 mg, from about 525 mg to about 675 mg, from about 550 mg to about 700 mg, from about 575 mg to about 725 mg, from about 600 mg to about 750 mg, from about 625 mg to about 775 mg, from about 650 mg to about 800 mg, from about 675 mg to about 825 mg, from about 700 mg to about 850 mg, from about 725 mg to about 875 mg, from about 750 mg to about 900 mg, from about 775 mg to about 925 mg, from about 800 mg to about 950 mg, from about 825 to about 975, from about 850 mg to about 1000 mg, from about 900 mg to about 1150 mg, from about 1000 mg to about 1150 mg, from about 1100 mg to about 1250 mg, and from about 1200 mg to about 1350 mg. A preferred oral dosage form has up to 600 mg of rodatristat ethyl, most preferably Form 3, taken twice per day (BID), for a total of up to 1200 mg per day. Another preferred oral dosage form has up to 300 mg of rodatristat ethyl, most preferably Form 3, taken twice per day, for a total of up to 600 mg per day. It is also possible to take these preferred dosage forms on a once-per-day (SID) basis.

The types of PAH treatable according to the methods disclosed herein include (1) idiopathic (IPAH), (2) familial (FPAH), and (3) associated (APAH) which is the most common type of PAH. The latter is PAH associated with other medical conditions including, for example, (1) collagen vascular disease (or connective tissue disease) which include autoimmune diseases such as scleroderma or lupus; (2) congenital heart and lung disease; (3) portal hypertension (e.g., resulting from liver disease); (4) HIV infection; and (5) drugs (e.g., appetite suppressants, cocaine, and amphetamines). APAH can also be PAH associated with abnormal narrowing in the pulmonary veins and/or capillaries such as in pulmonary veno-occlusive disease (PVOD) and pulmonary capillary hemangiomatosis. Another type of PAH is associated with persistent pulmonary hypertension of the newborn (PPHN).

Dosages/compositions according to the present disclosure include combinations of rodatristat ethyl and/or rodatristat with one or more of the other FDA-approved PAH pharmaceutical actives disclosed above. Preferred dosages/compositions are combinations of rodatristat ethyl and the other PAH pharmaceutical actives. A most preferred combination is rodatristat ethyl and selexipag. Oral dosage forms are preferred, although other forms of administration, e.g., parenteral, are possible.

A preferred dosage regimen of rodatristat ethyl and selexipag takes the form of up to 1200 mg of rodatristat ethyl and up to 2400 mcg of selexipag per day. A more preferred dosage regimen is up to 600 mg of rodatristat ethyl and up to 1200 mcg of selexipag twice per day. A still more preferred embodiment is about 200 mg to 600 mg of rodatristat ethyl and about 200 mcg to about 1200 mcg of selexipag twice per day. A most preferred embodiment is about 400 mg to 600 mg of rodatristat ethyl and about 200 mcg to about 1200 mcg of selexipag twice per day. An oral dosage regimen is preferred. The rodatristat ethyl and selexipag can be administered in the same dosage form or in separate dosage forms.

A preferred dosage regimen of rodatristat ethyl and sildenafil citrate takes the form of up to 1200 mg of rodatristat ethyl and up to 60 mg of sildenafil citrate per day. A more preferred dosage regimen is up to 600 mg of rodatristat ethyl and up to 30 mg of sildenafil citrate twice per day. A still more preferred embodiment is about 200 mg to 600 mg of rodatristat ethyl and about 10 mg to about 30 mg of sildenafil citrate twice per day. A most preferred embodiment is about 400 mg to 600 mg of rodatristat ethyl and about 20 mg to about 30 mg of sildenafil citrate twice per day. An oral dosage regimen is preferred. The rodatristat ethyl and sildenafil citrate can be administered in the same dosage form or in separate dosage forms.

A preferred dosage regimen of rodatristat ethyl and tadalafil takes the form of up to 1200 mg of rodatristat ethyl and up to 40 mg of tadalafil per day. A more preferred dosage regimen is up to 600 mg of rodatristat ethyl and up to 20 mg of tadalafil twice per day. A still more preferred embodiment is about 200 mg to 600 mg of tadalafil and about 6.67 mg to about 20 mg of tadalafil twice per day. A most preferred embodiment is about 400 mg to 600 mg of rodatristat ethyl and about 13.33 mg to about 20 mg of tadalafil twice per day. An oral dosage regimen is preferred. The rodatristat ethyl and tadalafil can be administered in the same dosage form or in separate dosage forms.

A preferred dosage regimen of rodatristat ethyl and bosentan takes the form of up to 1200 mg of rodatristat ethyl and up to 250 mg of bosentan per day. A more preferred dosage regimen is up to 600 mg of rodatristat ethyl and up to 125 mg of bosentan twice per day. A still more preferred embodiment is about 200 mg to 600 mg of rodatristat ethyl and about 62.5 mg to about 125 mg of bosentan twice per day. A most preferred embodiment is about 400 mg to 600 mg of rodatristat ethyl and about 62.5 mg to about 125 mg of bosentan twice per day. An oral dosage regimen is preferred. The rodatristat ethyl and bosentan can be administered in the same dosage form or in separate dosage forms.

A preferred dosage regimen of rodatristat ethyl and ambrisentan takes the form of up to 1200 mg of rodatristat ethyl and up to 10 mg of ambrisentan per day. A more preferred dosage regimen is up to 600 mg of rodatristat ethyl and up to 5 mg of ambrisentan twice per day. A still more preferred embodiment is about 200 mg to 600 mg of rodatristat ethyl and about 2.5 mg to about 5 mg of ambrisentan twice per day. A most preferred embodiment is about 400 mg to 600 mg of rodatristat ethyl and about 2.5 mg to about 5 mg of ambrisentan twice per day. An oral dosage regimen is preferred. The rodatristat ethyl and ambrisentan can be administered in the same dosage form or in separate dosage forms.

A preferred dosage regimen of rodatristat ethyl and treprostinil takes the form of up to 1200 mg of rodatristat ethyl and up to 24 mg of treprostinil per day. A more preferred dosage regimen is up to 600 mg of rodatristat ethyl and up to 12 mg of treprostinil twice per day. A still more preferred embodiment is about 200 mg to 600 mg of treprostinil and about 4 mg to about 12 mg of treprostinil twice per day. A most preferred embodiment is about 400 mg to 600 mg of rodatristat ethyl and about 8 mg to about 12 mg of treprostinil twice per day. An oral dosage regimen is preferred. The rodatristat ethyl and treprostinil can be administered in the same dosage form or in separate dosage forms.

A preferred dosage regimen of rodatristat ethyl and macitentan takes the form of up to 1200 mg of rodatristat ethyl and up to 10 mg of macitentan per day. A more preferred dosage regimen is up to 600 mg of rodatristat ethyl and up to 5 mg of macitentan twice per day. A still more preferred embodiment is about 200 mg to 600 mg of macitentan and about 2.5 mg to about 5 mg of macitentan twice per day. A most preferred embodiment is about 400 mg to 600 mg of rodatristat ethyl and about 2.5 mg to about 5 mg of macitentan twice per day. An oral dosage regimen is preferred. The rodatristat ethyl and macitentan can be administered in the same dosage form or in separate dosage forms.

It is also possible to administer combinations of rodatristat ethyl or rodatristat with two or more of the other PAH actives. For instance, a useful combination is a three-drug combination of rodatristat ethyl/tadalafil/ambrisentan.

Combinations of rodatristat and/or rodatristat ethyl with one or more of the other FDA-approved PAH pharmaceutical actives are formulated such that one or more of the following are accomplished: (A) the risk of DDI interactions is low, (B) synergistic or additive reduction in symptoms of PAH compared to the rodatristat/rodatristat ethyl and the FDA-approved PAH pharmaceutical active(s) alone, and (C) synergistic or additive reduction in side effects of PAH compared to the rodatristat/rodatristat ethyl and the FDA-approved PAH pharmaceutical active(s) alone.

Symptoms of PAH include the following: fatigue, lethargy, exertional dyspnea, presyncope/syncope, cough, hoarseness, hypotension, fluid retention, lower extremity edema, chest pain, and cyanosis.

Side effects of PAH include the following: the aforementioned symptoms, side effects of multi-drug treatment regimens and diminished quality of life for the patient and family caregivers.

In an embodiment of the disclosure, there is a method for treating pulmonary arterial hypertension, comprising administering daily to a human patient in need thereof a therapeutic dose of (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing, wherein the rodatristat ethyl and its active metabolite rodatristat exhibit an IC₅₀ of 30 μM for one or more of the enzyme group consisting of CYP1A2, CYP2C9, CYP2C19, and CYP2D6 in the bloodstream. IC₅₀ is measured according to the in vitro CYP inhibition method/technique using pooled human microsomes.

In an embodiment of the disclosure, there is a method for treating pulmonary arterial hypertension, comprising administering daily to a human patient in need thereof a therapeutic dose of (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing, wherein the rodatristat ethyl and its active metabolite rodatristat exhibit an IC₅₀ of 30 μM for one or more of the enzyme group consisting of CYP1A2, CYP2B6, CYP2C9, CYP2C19, CYP2D6, and CYP3A4. IC₅₀ is measured according to the in vitro CYP inhibition method/technique using pooled human microsomes.

The subject matter of U.S. Provisional Application No. 62/968,651, filed Jan. 31, 2020, is incorporated herein by reference in its entirety.

The disclosure is further illustrated herein by the following non-limiting examples.

### EXAMPLES

### Example 1

### Rodatristat Ethyl: Assessment of Potential for Metabolism or Transporter-Based Drug-Drug Interactions (DDIs) and Contextualization to Approved Medications for Pulmonary Arterial Hypertension

The potential for rodatristat ethyl or rodatristat to be perpetrators of drug-drug interactions was assessed across in vitro CYP and transporter studies (Tables 1-4). Where appropriate, IC₅₀ values were compared to the predicted mean maximum plasma concentrations (Cₘₐₓ) of rodatristat ethyl (149 ng/mL, 0.252 μM) and rodatristat (1130 ng/mL, 2.01 μM) in plasma ELEVATE1 and highest dose level in ELEVATE2 (600 mg rodatristat ethyl dosed bid). This analysis was performed to assess the potential for clinically meaningful DDIs as recommended in FDA Draft Guidance: “In Vitro Metabolism- and Transporter-Mediated Drug-Drug Interaction Studies Guidance for Industry”; October 2017 (FDA, 2017).

This analysis is typically based on non-protein bound drug levels in plasma. Values for plasma protein binding (fraction bound) of rodatristat ethyl and rodatristat were 0.995 and 0.991, respectively. Unbound drug concentrations were <1% for both compounds, consequently per FDA recommendation a conservative 1% free fraction was used to calculate the unbound Cₘₐₓ concentrations of 0.0025 μM and 0.0201 μM, for rodatristat ethyl and rodatristat, respectively.

Instances where the in vitro studies suggest potential are discussed with context to approved PAH medications.

### In Vitro Characterization of Potential for Metabolism Based Interactions

Rodatristat ethyl and rodatristat are not major substrates for key CYP enzymes and are not time-dependent inhibitors of CYP isoforms evaluated.

### CYP Inhibition by Rodatristat Ethyl:

The metabolism of rodatristat ethyl is predominantly by esterase mediated hydrolysis of the ester pro-drug moiety to rodatristat in liver tissue preparations from human, rat, dog, and cynomolgus monkey, and intestinal and lung tissues from rat and human. Hydrolysis of rodatristat ethyl was also observed in rat blood, and to a much lesser extent in dog and human blood. Rodatristat is metabolically stable in liver preparations across species and is largely eliminated unchanged in bile (rat).

Rodatristat ethyl directly inhibited CYP2B6, CYP2C8, and CYP3A4/5 (for both midazolam 1′ hydroxylation and testosterone 6β hydroxylation) with IC50 values of 13 μM, 2.8 μM, 13 μM, and 23 μM, respectively. There was no evidence of clinically meaningful direct inhibition of CYP1A2, CYP2C9, CYP2C19, or CYP2D6 by rodatristat ethyl as the IC50 values were greater than 30 μM. The predicted clinical Cₘₐₓ for rodatristat ethyl for the proposed 600 mg BID regimen is 0.252 μM, which is ˜10 fold lower than the IC₅₀ for the most sensitive CYP isoform 2C8 (IC50 2.8 μM), indicating rodatristat ethyl has low potential to cause a CYP2C8 based interaction.

Inhibition of CYP3A, while low risk systemically, does meet threshold for potential intestinal interaction. This is determined from the dose and estimated exposure in the gut lumen versus Ki (R1,gut). The R1 value calculated for midazolam is >600 and exceeds the threshold of 11.

### CYP Inhibition by Rodatristat:

Cytochrome (CYP) inhibition was evaluated using a pool (n=200) of human liver microsomes incubated with rodatristat ethyl (RE) or rodatristat (R) concentrations of up to 30 mM (˜120×RE Cₘₐₓ and ˜15×R Cₘₐₓ). “mM^{”}=millimolar. CYP induction was evaluated using cultured human hepatocytes (from 3 donors) treated once daily for 3 days with RE/R concentrations to 20 mM.

Rodatristat elicited weak inhibition of CYPs 2B6, 2C9, and 3A4/5, with 27 to 45% inhibition across these isoforms at 30 μM rodatristat, indicating IC₅₀ values are >30 μM and >14× the envisaged rodatristat clinical Cₘₐₓ (2.01 μM) at steady state. Rodatristat did not inhibit CYP1A2, CYP2C19, CYP2D6.

Rodatristat directly inhibited CYP2C8 with an IC₅₀ value of 2.8 μM. The FDA guidance recommends contextualizing IC₅₀ values relative to unbound Cₘₐₓ values in plasma. The ratio (0.014) of unbound Cₘₐₓ (0.02 μM) to Ki (1.4 μM, equivalent to IC50/2) is used to calculate an R value (R=1+unbound Cₘₐₓ/Ki) where a value ≥1.02 is considered predictive for a clinically-meaningful interaction. The R value for rodatristat is 1.014 supportive of low risk for a clinically meaningful interaction via CYP2C8.

### CYP Induction Potential:

CYP induction was evaluated in vitro using cultured human hepatocytes from 3 donors. Rodatristat ethyl and rodatristat caused negligible induction of CYP1A2 (<2% control for the most sensitive responder), CYP2B6 (mean±SD values relative to control of 16.2±20.0% and 15.0±17.9%, respectively), and CYP3A4 (7.1±6.41% and 10.3±11.9%, respectively) mRNA at 20 μM (the highest concentration studied). The 20 μM concentration reflects at least ˜9-fold the predicted clinical Cₘₐₓ values predicted for the 600 mg BID dose for rodatristat ethyl (0.252 μM) and rodatristat (2.01 μM). The highest values for induction of CYP2B6 by an individual donor at all concentrations tested were 60.1% and 57.4% at 10 μM for rodatristat ethyl and rodatristat, respectively, and for CYP3A4 were 13.3% and 23.0% at 20 μM, respectively. Overall, rodatristat ethyl and rodatristat are not considered inducers of CYP1A2, potentially weak to moderate inducers for CYP2B6, and potential weak inducers for CYP3A4 at clinically relevant concentrations.

Evaluation of in vitro data (Tables 1 and 2) indicates potential for perpetrator interactions by rodatristat ethyl or rodatristat on 2B6 and 3A induction or inhibition of gut 3A. The likely extent of these potential interactions has been evaluated by PBPK modeling.

### In Vitro Characterization of Potential for Drug Transporter Based Interactions

Transporter interaction data and potential for interactions are summarized in Tables 3 and 4.

Perpetrator Interactions: Rodatristat ethyl and rodatristat inhibited OATP1B1 and OATP1B3 where IC50 values for OATP1B1 are 25.4 μM and 5.74 μM for rodatristat ethyl and rodatristat, respectively, and 12.3 μM and 1.93 μM for OATP1B3. The R values FDA guidance for rodatristat ethyl with OATP1B1 and OATP1B3, and rodatristat with OATP1B1 are <1.1 indicating low potential for a clinically relevant interaction. The R value for rodatristat is 1.26 for OATP1B3, indicating a potential for an interaction. However, overlap of drug substrates for OATPs 1B1 and 1B3 suggests low overall risk for interactions with statins.

Rodatristat ethyl and rodatristat demonstrate little potential to inhibitor OCT2 at concentrations of up to 20 μM (IC50 values >20 μM). Rodatristat weakly inhibited multidrug resistance-associated protein 2 (MRP2; by 25%) and multidrug and toxin extrusion (MATE) 1 (by 30%) at nominal concentrations of 30 μM. Rodatristat did not inhibit MATE2-K (up to 30 μM nominal concentration). Neither rodatristat ethyl nor rodatristat inhibited OAT1, OAT3, and breast cancer resistance protein (BCRP). Thus, rodatristat ethyl and rodatristat are not anticipated to alter the pharmacokinetics of therapeutic agents that are substrates for BCRP, OAT1, OAT3, and OCT2.

Rodatristat ethyl is an inhibitor of P-gp in Caco 2 cells (IC50=3.32 μM; Igut/IC50 >>10), but not an inhibitor of BCRP. Rodatristat was not an inhibitor of P-gp or BCRP. Systemic Cₘₐₓ levels of rodatristat and rodatristat ethyl are not expected to elicit systemic drug interactions. Rodatristat ethyl may alter the pharmacokinetics of substrates of P-gp by inhibition of intestinal P-gp.

Victim Interactions: Rodatristat ethyl is not a substrate for P-gp, BCRP, MRP2, OATP1B1, or OATP1B3. Rodatristat is not an in vitro substrate of MRP2, or MATE1. Rodatristat is an in vitro substrate of the OATP1B1 (2.22-fold accumulation at 3 μM) and OATP1B3 (9.28-fold accumulation at 3 μM) uptake transporters. Rodatristat is a potential substrate for P-gp and BCRP; however, owing to the very low passive permeability and compound recovery in Caco 2 and MDCK cell studies, it was not possible to obtain an accurate estimate of basolateral to apical (B A) versus apical to basolateral (A B) efflux ratios. Consequently, the elimination of rodatristat into bile may be impacted by co-administration with agents that are BCRP inhibitors.

The potential for clinically-meaningful interactions with OATP1B1/3 and P-gp has been evaluated by PBPK modeling.

### Potential Drug-Drug Interactions Between Rodatristat Ethyl and Standard of Care Medications in PAH

In vitro CYP450 and transporter interaction data were used to assess potential for systemic DDIs with PAH medications using information including Forest plots in product monographs. Neither rodatristat ethyl nor rodatristat exceeded criteria in the FDA guidance predictive of systemic clinically meaningful drug interactions with CYP isoforms. It should be noted that most approved PAH medications are metabolized primarily by CYP3A or CYP2C9, and CYP2C8 for treprostinil and selexipag. These isoforms have little role in metabolism of rodatristat ethyl or rodatristat. Rodatristat is metabolically-stable and unlikely to be impacted to a clinically-relevant extent by CYP inhibitors.

Rodatristat ethyl and rodatristat were weak in vitro inducers in CYPs 2B6 and 3A in human hepatocytes. Monographs of PAH medications indicate CYP2B6 is not a key metabolizing enzyme. In vitro induction of CYP3A is low and unlikely to warrant dose-adjustments.

The in vitro data for rodatristat ethyl nor rodatristat are contextualized for key approved PAH medications below. Epoprostenol is not discussed as pharmacokinetic drug interaction studies have not been reported owing to its very short half-life and lack of bioanalytical methods to determine levels in plasma.

Treprostinil and Selexipag: Treprostinil and Selexipag are primarily metabolized by CYP2C8. This isoform is inhibited by rodatristat and the potential for interaction warrants further consideration as both drugs are considered sensitive to being victims of interactions as their doses are typically titrated to effect or the highest tolerated. Inhibition of CYP2C8 by another medication could increase their exposure and the possibility of adverse events.

Walsky, et alⁱ evaluated criteria to assess the potential for approved drugs to elicit clinically-meaningful drug interactions via inhibition of 2C8. Following their evaluation of 209 compounds they opined that unbound hepatic portal vein concentration (C_{max, u, portal}) compared to the in vitro Ki provides additional insight beyond peripheral Cₘₐₓ (used for FDA criteria) as it reflects the unbound drug concentration entering the liver.

Calculation of C_{max, u, portal} uses plasma Cₘₐₓ, fraction of an oral dose absorbed (Fa) and measured unbound fraction in blood. For rodatristat, experimentally derived values were used for Fa determined in a rat 14C study (20%) and in vitro binding to human plasma (99.1%) and blood:plasma distribution (R_{B}, 0.72). Walsky et al proposed a threshold where the ratio of C_{max, u, portal}/Ki of <0.1 is associated with low risk. In this analysis the C_{max, u, portal}/Ki for rodatristat was 0.128 reflecting low/moderate risk. However, this represents a worst-case situation as it does not consider that a higher ratio of rodatristat pro-drug (RE) to rodatristat (R) is present in portal blood compared to the circulation. In the rat, the RE:R is ˜3-fold higher in portal blood compared to the periphery. Thus, it is likely the C_{max, u, portal}/Ki ratio for rodatristat is <<0.128.

Clinical DDI studies have been performed for treprostinil or selexipag administered with gemfibrozil (strong inhibitor). Gemfibrozil is metabolized to an acyl glucuronide that is an irreversible metabolism-based inhibitor of CYP2C8 and likely exacerbates inhibitory effectsⁱⁱ. Studies were also performed for selexipag with clopidogrel (moderate inhibitor) or ritonavir (IC50, 3.03 μM and C_{max, u, portal}/Ki, 0.21; [Walsky et al]).

Selexipag: gemfibrozil increased exposure (AUC) of selexipag and its active metabolite (ACT-333679) by 2- and 11-fold, respectivelyⁱⁱⁱ. Coadministration with clopidogrel did not impact selexipag exposure but increased it by 2.7-fold for the active metabolite. Coadministration with ritonavir increased exposure of selexipag ˜2-fold and <2-fold for the metabolite. Selexipag administration is contraindicated in patients taking gemfibrozil and a lowering of daily dose (to QD) is recommended with moderate inhibitors. The comparison between ritonavir and rodatristat is helpful as they have similar IC50 values (3.03 μM and 2.8 μM, respectively) and ritonavir has a higher C_{max, u, portal}/Ki ratio (0.21 versus <0.128, respectively). Ritonavir did not increase exposure to the active metabolite to a clinically meaningful extent supportive that treatment with rodatristat ethyl should not require a reduction in selexipag dose.

Selexipag and its metabolite are in vitro substrates for P-gp, BCRP, OAT1B1 and OAT1B3. Rodatristat is an inhibitor of OAT1B3 but not OAT1B1, thus hepatic uptake may be unaffected.

Treprostinil: Treprostinil is available in 3 formulations and 4 different routes of administration (PO, IV, SC and inhaled). IV and SC are considered bioequivalent at steady-state, inhaled achieves lower systemic exposure while delivering higher concentrations locally in lung and oral treprostinil achieves similar exposure to IV/SC with bioavailability of ˜17%^{iv}. The oral route likely presents the greater risk for potential DDI potential owing to the comparatively higher portal drug concentrations.

Treprostinil is metabolized primarily by CYP2C8 and to a lesser extent CYP2C9. Neither rodatristat ethyl nor rodatristat inhibit CYP2C9. Coadministration with gemfibrozil increased treprostinil Cₘₐₓ and AUC by ˜2-fold. Studies do not appear to have been performed with moderate CYP2C8 inhibitors probably because the magnitude of the interaction would be less than for gemfibrozil and not clinically meaningful. Likewise, it is unlikely that rodatristat ethyl treatment would cause meaningful exposure changes. Consequently, rodatristat ethyl and oral treprostinil can be co-administered without dose adjustments but with appropriate clinical monitoring with a focus on changes in tolerability to treprostinil.

A similar strategy was reported by the PH Professional Network^{v} for PAH patients with HIV and/or HCV where they recommend “No dose adjustments. Monitor therapy” for coadministration of treprostinil with efavirenz (CYP2C8 IC50, 4.80 μM^{vi}) or ritonavir.

Ambrisentan: Studies with human liver tissue indicate that ambrisentan is metabolized by CYP3A, CYP2C19, and uridine 5′-diphosphate glucuronosyltransferases (UGTs) 1A9S, 2B7S, and 1A3S (Letairis (ambrisentan) [package insert], 2018).

Across multiple clinical drug-drug interaction studies, a clinically relevant interaction has been demonstrated only with cyclosporine. Of note, no dose adjustment was required in perpetrator studies with digoxin, ritonavir, or warfarin. Similarly, no dose adjustment was indicated in victim studies with rifampin, ritonavir, ketoconazole or tacrolimus. Consequently, the risk of a clinically relevant drug interaction with rodatristat ethyl is considered low because ambrisentan is not metabolized by CYP2C8, did not require a dose adjustment when administered with the CYP3A inducer Rifampin and is not impacted by P-gp inhibitors or CYP3A inhibitors or inducers.

Bosentan: Bosentan is metabolized by CYPs 2C9 and 3A4 and is consequently unlikely to be impacted by rodatristat ethyl or rodatristat (Tracleer (bosentan) [package insert], 2018). No dose adjustment was required when co-administered with the CYP3A inducer rifampin, nor ketoconazole indicating low risk of a CYP3A4-mediated or a P-gp mediated interaction with rodatristat ethyl.
Sildenafil: Sildenafil is metabolized primarily by CYP3A (major route) and 2C9 (minor route) and is consequently unlikely to be impacted by rodatristat ethyl or rodatristat (Revatio (sildenafil) [package insert], 2018). No dose adjustment was needed when sildenafil was co-administered with the CYP3A inducer bosentan, nor squinavir, suggesting a CYP3A4-mediated or P-gp-mediated interaction with rodatristat ethyl is unlikely.
Tadalafil: Tadalafil is metabolized primarily by CYP3A4 and is consequently unlikely to have metabolism-based interactions with rodatristat ethyl or rodatristat (Adcirca (tadalafil) [package insert], 2017). No dose adjustment is required when co-administered with weak to moderate CYP3A inducers. Tadalafil did not have a significant impact on the pharmacokinetics of digoxin.

TABLE 1
Rodatristat ethyl DDI Risk Assessment (predicted @ 600 mg BID): Metabolism

| | Perpetrator Potential | | | | | | | | | | | | |
|---|---|---|---|---|---|---|---|---|---|---|---|---|---|
| | | | Time-Dependent | | | Induction | | | Potential Clinical Interaction | | | Victim Potential | |
| | Direct Inhibition | | Inhibitor (TDI) | | | In Vitro | | Cal- | Cal- | Potential | Clinical | | Clinical |
| CYP | In vitro | Calculated | Inactivation | | Calculated | induction | | culated | culated | for | DDI study | In vitro | DDI study |
| Enzyme | IC50 (μM) | R1 Value* | parameters | | R2 Value* | parameters | | R3 Value | AUCR* | interaction | conducted? | substrate? | conducted? |
| CYP1A2 | 0 | NC | K [symbol] | ND | <1.25 | EC [symbol] | ND | NC | NC | No | No | No | No |
| | | | K [symbol] | ND | | E [symbol] | ND | | | | | | |
| CYP2B6 | 13 | <1.02 | K [symbol] | ND | <1.25 | EC [symbol] | 6.16 | 0.960 | 0.511 | Yes | Should | No | No |
| | | | K [symbol] | ND | | E [symbol] | 3.1 | | | | consider | | |
| CYP2C8 | 2.8 | <1.02 | K [symbol] | ND | <1.25 | EC [symbol] | ND | NC | NC | No | No | No | No |
| | | | K [symbol] | ND | | E [symbol] | ND | | | | | | |
| CYP2C9 | 0 | NC | K [symbol] | ND | <1.25 | EC [symbol] | ND | NC | NC | No | No | No | No |
| | | | K [symbol] | ND | | E [symbol] | ND | | | | | | |
| CYP2C19 | 0 | NC | K [symbol] | ND | <1.25 | EC [symbol] | ND | NC | NC | No | No | No | No |
| | | | K [symbol] | ND | | E [symbol] | ND | | | | | | |
| CYP2D6 | 0 | NC | K [symbol] | ND | <1.25 | EC [symbol] | ND | NC | NC | No | No | No | No |
| | | | K [symbol] | ND | | E [symbol] | ND | | | | | | |
| CYP3A4 | 13 | <1.02 | K [symbol] | ND | <1.25 | EC [symbol] | 15.6 | 0.925 | 0.510 | Yes | Should | No | No |
| (M) | | R1gut = 627 | K [symbol] | ND | | E [symbol] | 4.53 | | | | consider | | |
| CYP3A4 | 23 | <1.02 | K [symbol] | ND | <1.25 | EC [symbol] | 15.6 | 0.925 | 0.298 | | | | |
| (T) | | R1gut = 355 | K [symbol] | ND | | E [symbol] | 4.53 | | | | | | |

[symbol]

indicates data missing or illegible when filed

TABLE 2
Rodatristat DDI Risk Assessment (predicted @ 600 mg BID): Metabolism

| | Perpetrator Potential | | | | | | | | | | | | |
|---|---|---|---|---|---|---|---|---|---|---|---|---|---|
| | | | Time-Dependent | | | Induction | | | Potential Clinical Interaction | | | Victim Potential | |
| | Direct Inhibition | | Inhibitor (TDI) | | | In Vitro | | Cal- | Cal- | Potential | Clinical | | Clinical |
| CYP | In vitro | Calculated | Inactivation | | Calculated | induction | | culated | culated | for | DDI study | In vitro | DDI study |
| Enzyme | IC50 (μM) | R1 Value* | parameters | | R2 Value* | parameters | | R3 Value | AUCR* | interaction | conducted? | substrate? | conducted? |
| CYP1A2 | 0 | NC | K [symbol] | ND | <1.25 | EC [symbol] | ND | NC | NC | No | No | No | No |
| | | | K [symbol] | ND | | E [symbol] | ND | | | | | | |
| CYP2B6 | 0 | NC | K [symbol] | ND | <1.25 | EC [symbol] | 5.6 | 0.766 | 0.464 | Yes | Should | No | No |
| | | | K [symbol] | ND | | E [symbol] | 2.95 | | | | consider | | |
| CYP2C8 | 2.8 | <1.02 | K [symbol] | ND | <1.25 | EC [symbol] | ND | NC | NC | No | No | No | No |
| | | | K [symbol] | ND | | E [symbol] | ND | | | | | | |
| CYP2C9 | 0 | NC | K [symbol] | ND | <1.25 | EC [symbol] | ND | NC | NC | No | No | No | No |
| | | | K [symbol] | ND | | E [symbol] | ND | | | | | | |
| CYP2C19 | 0 | NC | K [symbol] | ND | <1.25 | EC [symbol] | ND | NC | NC | No | No | No | No |
| | | | K [symbol] | ND | | E [symbol] | ND | | | | | | |
| CYP2D6 | 0 | NC | K [symbol] | ND | <1.25 | EC [symbol] | ND | NC | NC | No | No | No | No |
| | | | K [symbol] | ND | | E [symbol] | ND | | | | | | |
| CYP3A4 | 0 | NC | K [symbol] | ND | <1.25 | EC [symbol] | 26.7 | 0.476 | 0.015 | Yes | Should | No | No |
| (M) | | R1gut = NC | K [symbol] | ND | | E [symbol] | 4.5 | | | | consider | | |
| CYP3A4 | 0 | NC | K [symbol] | ND | <1.25 | EC [symbol] | 26.7 | 0.476 | 0.184 | | | | |
| (T) | | R1gut = NC | K [symbol] | ND | | E [symbol] | 4.5 | | | | | | |

[symbol]

indicates data missing or illegible when filed

TABLE 3
Rodatristat ethyl DDI Risk Assessment (predicted @ 600 mg BID): Transporters

| Transporter Inhibitor | | | | | | Transporter Substrate | |
|---|---|---|---|---|---|---|---|
| | | | | | | | Clinical |
| | In vitro IC₅₀ | FDA | Calculated | Potential for | Clinical Interaction | In vitro | Interaction |
| Transporter | (μM) | Equation | Values* | interaction? | Study? | substrate? | Study? |
| OATP1B1 | 25.4 | R-value | <1.1 | No | No | No | No |
| OATP1B3 | 12.3 | R-value | <1.1 | No | No | No | No |
| OAT1 | 0 | [symbol] | NC | No | No | ND | No |
| OAT3 | 0 | [symbol] | NC | No | No | ND | No |
| OCT2 | 0 | [symbol] | NC | No | No | NO | No |
| MATE1 | 0 | [symbol] | NC | No | No | ND | No |
| MATE2-K | 0 | [symbol] | NC | No | No | ND | No |
| MRP2 | 0 | [symbol] | NC | No | No | not likely | No |
| P-gp | 3.32 | [symbol] | 1225 | Yes | Should consider | not likely | No |
| BCRP | 0 | [symbol] | NC | No | No | not likely | No |

[symbol]

indicates data missing or illegible when filed

TABLE 4
Rodatristat DDI Risk Assessment (predicted @ 600 mg BID): Transporters

| Transporter Inhibitor | | | | | | Transporter Substrate | |
|---|---|---|---|---|---|---|---|
| | | | | | | | Clinical |
| | In vitro IC₅₀ | FDA | Calculated | Potential for | Clinical Interaction | In vitro | Interaction |
| Transporter | (μM) | Equation | Values* | interaction? | Study? | substrate? | Study? |
| OATP1B1 | 5.74 | R-value | <1.1 | No | No | Yes | should |
| | | | | | | | consider |
| OATP1B3 | 1.93 | R-value | 1.26 | Yes | Should consider | Yes | should |
| | | | | | | | consider |
| OAT1 | 0 | [symbol] /IC₅₀ | NC | No | No | ND | No |
| OAT3 | 0 | [symbol] /IC₅₀ | NC | No | No | ND | No |
| OCT2 | 0 | [symbol] /IC₅₀ | NC | No | No | ND | No |
| MATE1 | 0 | [symbol] /IC₅₀ | NC | No^{a} | No | No | No |
| MATE2-K | 0 | [symbol] /IC₅₀ | NC | No | No | ND | No |
| MRP2 | 0 | [symbol] /IC₅₀ | NC | No^{a} | No | Not likely | No |
| P-gp | 0 | [symbol] /IC₅₀ | NC | No | No | See footnote^{b} | No |
| BCRP | 0 | [symbol] /IC₅₀ | NC | No | No | See footnote^{b} | No |

[symbol]

indicates data missing or illegible when filed

Overall, these data support coadministration of selexipag and rodatristat ethyl without dose adjustment, but with appropriate clinical monitoring with a focus on changes in tolerability to selexipag.

### FDA Guidance Risk Assessment

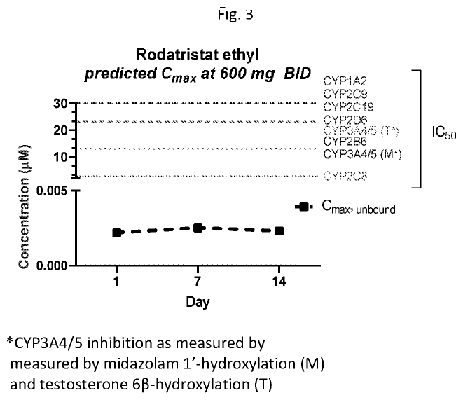

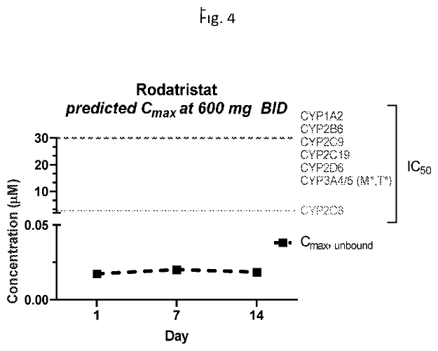

Risk assessment was performed following the FDA guidance IC₅₀ values (half-maximal inhibitory concentration) were compared to the predicted maximum plasma concentrations (Cₘₐₓ) of RE (149 ng/mL, 0.252 μM) and R (1130 ng/mL, 2.01 μM) in plasma for ELEVATE1 and the highest dose in ELEVATE2 studies for patients receiving a 600 mg RE twice-daily (BID) regimen. R1 and R3 values using free (non-protein bound) drug levels were calculated based on plasma protein binding (fraction bound, fb) of rodatristat ethyl (0.995) and rodatristat (0.991). The FDA Guidance is set forth In Vitro Metabolism- and Transporter-Mediated Drug-Drug Interaction Studies Guidance for Industry (2017). Per FDA guidance, when fb >0.99, a conservative 1% free fraction was used to calculate the unbound Cₘₐₓ concentrations of 0.0025 μM and 0.0201 μM, for rodatristat ethyl and rodatristat, respectively. Rodatristat, the active primary metabolite is produced by carboxylesterases and metabolically stable supporting low risk for victim PK interactions. Rodatristat ethyl and rodatristat are not time-dependent CYP inhibitors. Overall, in vitro data indicate modest potential for perpetrator interactions by RE or R on CYP2B6 and CYP3A induction or inhibition of gut CYP3A—this will be evaluated with physiologically-based pharmacokinetic modeling. As shown in FIGS. 3 and 4, the predicted Cₘₐₓ for the 600 mg BID dose for RE and R predicts low risk for CYP inhibition. Parameters for the prediction are set forth below in Tables 5 and 6.

TABLES 5 and 6
FDA Guidance-Based Risk Assessment for the 600 mg BID rodatristat Ethyl Dose

| | [symbol] | | | | | | | |
|---|---|---|---|---|---|---|---|---|
| | | | | | | [symbol] | | |
| | [symbol] | | [symbol] | | | Cal- | Potential | Clinical |
| CYP | [symbol] | [symbol] | [symbol] | | Calculated | culated | for | DDI study |
| Enzyme | [symbol] | [symbol] | [symbol] | | R3 Value | AUCR* | interaction | conducted? |
| CYP1A2 | 0 | NC | EC [symbol] | ND | NC | NC | No | No |
| | | | E [symbol] | ND | | | | |
| CYP2B6 | 13 | <1.02 | EC [symbol] | 8.16 | [symbol] | [symbol] | Yes | Should |
| | | | E [symbol] | 3.1 | | | | consider |
| CYP2C8 | 2.8 | <1.02 | EC [symbol] | ND | NC | NC | No | No |
| | | | E [symbol] | ND | | | | |
| CYP2C9 | 0 | NC | EC [symbol] | ND | NC | NC | No | No |
| | | | E [symbol] | ND | | | | |
| CYP2C19 | 0 | NC | EC [symbol] | ND | NC | NC | No | No |
| | | | E [symbol] | ND | | | | |
| CYP2D6 | 0 | NC | EC [symbol] | ND | NC | NC | No | No |
| | | | E [symbol] | ND | | | | |
| CYP3A4 | 13 | <1.02 | EC [symbol] | [symbol] | 0.478 | 0.015 | Yes | Should |
| (M) | | R1gut = 627 | E [symbol] | [symbol] | | | | consider |
| CYP3A4 | 23 | <1.02 | EC [symbol] | [symbol] | 0.478 | 0.164 | | |
| (T) | | R1gut = 355 | E [symbol] | [symbol] | | | | |

FDA Guidance-Based Risk Assessment for the 600 mg BID rodatristat Ethyl Dose

| | [symbol] | | | | | | | |
|---|---|---|---|---|---|---|---|---|
| | | | | | | [symbol] | | |
| | [symbol] | | [symbol] | | | Cal- | Potential | Clinical |
| CYP | [symbol] | [symbol] | [symbol] | Calculated | | culated | for | DDI study |
| Enzyme | [symbol] | [symbol] | [symbol] | R3 Value | | AUCR* | interaction | conducted? |
| CYP1A2 | 0 | NC | EC [symbol] | ND | NC | NC | No | No |
| | | | E [symbol] | ND | | | | |
| CYP2B6 | 0 | NC | EC [symbol] | [symbol] | [symbol] | [symbol] | Yes | Should |
| | | | E [symbol] | [symbol] | | | | consider |
| CYP2C8 | 2.8 | <1.02 | EC [symbol] | ND | NC | NC | No | No |
| | | | E [symbol] | ND | | | | |
| CYP2C9 | 0 | NC | EC [symbol] | ND | NC | NC | No | No |
| | | | E [symbol] | ND | | | | |
| CYP2C19 | 0 | NC | EC [symbol] | ND | NC | NC | No | No |
| | | | E [symbol] | ND | | | | |
| CYP2D6 | 0 | NC | EC [symbol] | ND | NC | NC | No | No |
| | | | E [symbol] | ND | | | | |
| CYP3A4 | 0 | NC | EC [symbol] | [symbol] | [symbol] | [symbol] | Yes | Should |
| (M) | | R1gut = NC | E [symbol] | [symbol] | | | | consider |
| CYP3A4 | 0 | NC | EC [symbol] | [symbol] | [symbol] | [symbol] | | |
| (T) | | R1gut = NC | E [symbol] | [symbol] | | | | |

[symbol]

indicates data missing or illegible when filed

R1=1+(I_{max, unbound}/Ki)
R3=1/[1+(d×Eₘₐₓ×10×I_{max,u})/(EC₅₀+(10×I_{max,u}))]
I_{max,u} is the Cₘₐₓ of the unbound interacting drug
Kᵢ is the unbound inhibition constant determined in vitro
Eₘₐₓ is maximum induction effect determined in vitro
EC₅₀ is the concentration causing half-maximal effect determined in vitro
AUCR calculation is disclosed in the FDA Guidance
No time-dependent inhibition was observed for rodatristat ethyl or rodatristat.

### Findings

Rodatristat ethyl and rodatristat are low risk for being systemic inhibitors of CYPs. The predicted unbound Cₘₐₓ for RE and R >100-fold lower than IC₅₀ for most sensitive CYP isoform 2C8 (IC50 2.8 μM).

### Select FDA-Approved PAH Pharmaceutical Actives+Rodatristat Ethyl: A DDI Risk Overview

Treprostinil and selexipag: -sensitive CYP2C8 substrates; rodatristat inhibits CYP2C8 in vitro but presents a low-risk for clinically meaningful interaction.

Treprostinil: coadministration with strong inhibitor gemfibrozil increased Cₘₐₓ and AUC by ˜2-fold (ORENITRAM® (treprostinil) monograph (revised 10/2019) haps://www.accessdata.fda.gov/drugsatida_docs/label/2019/203496s011lbl.pdf. As a weaker inhibitor, it is unlikely that RE treatment would cause meaningful exposure changes. Treprostinil can be co-administered without dose adjustments but with appropriate clinical monitoring with a focus on changes in tolerability to treprostinil.

Selexipag: lowering of daily dose (to QD) is recommended with moderate inhibitors. See UPTRAVI® (selexipag) monograph (revised 09/2019) https://www.accessdata.fda.gov/drugsatida_docs/label/2019/207947s007lbl.pdf. No dose adjustment is required with ritonavir (2C8 IC₅₀, 3.03 μM⁵) suggesting low risk for clinically meaningful interaction for RE co-administration with selexipag. See Itkonen, M. K., Tornio, A., Neuvonen, M., Neuvonen, P. J., Niemi, M., and Backman, J. T, 2019. Drug Metab Dispos. 47(4). 377-385. Coadministration should be performed with appropriate clinical monitoring with a focus on changes in tolerability to selexipag.

Ambrisentan: metabolized by CYPs 3A4, 2C19, UGTs 1A9S, 2B7S, 1A3S and is unlikely to require dose adjustment with RE. See LETAIRIS ° (ambrisentan) monograph (revised 08/2019) haps://www.accessdata.fda.gov/drugsatida_docs/label/2019/022081s041lbl.pdf.

Bosentan: metabolized by CYPs 2C9 and 3A4 and is unlikely to require dose adjustment with RE TRACLEER (bosentan) monograph (revised 05/2019) https://www.accessdata.fda.gov/drugsatfda_docs/label/2019/021290s039,209279s005lbl.pdf.

Tildenafil: metabolized by CYPs 3A (major route) and 2C9 (minor route) and is unlikely to require dose adjustment with RE⋅ See REVATIO® (sildenafil) monograph (revised 02/2018) haps://www.accessdata.fda.gov/drugsatida_docs/label/2018/021845s018lbl.pdf.

Tadalafil: metabolized primarily by CYP3A4 and is unlikely to require dose adjustment with RE. See ADCIRCA (tadalafil) monograph (revised 05/2017) <https://www.accessdata.fda.gov/drugsatfda_docs/label/2017/022332s009lbl.pdf>

### Example 2

### Summary

A ‘fit-for-purpose’ physiologically based pharmacokinetic (PBPK) model was developed for rodatristat ethyl and rodatristat, based on available physiochemical, in vitro and clinical PK data. This PBPK model was then applied to predict rodatristat ethyl and rodatristat plasma concentration-time profiles after single-dose and multiple-dose oral administration in fed healthy volunteers.

The PBPK model was developed based on physicochemical data, in vitro data, and refined using clinical data collected in Clinical Study RVT-1201-1001 (Regimen C; single 400 mg dose in fed subjects). The PBPK model was then verified by simulating the multiple-dose plasma concentration-time profile of rodatristat ethyl and rodatristat after twice daily (BID) administration of 400 mg (fed) or 800 mg (fed) rodatristat ethyl in Clinical Study RVT-1201-1001. In addition, the PBPK model was further verified by simulating of the multiple-dose BID administration of 400 mg (fed) rodatristat ethyl in Clinical Study KAR5585-101. In each verification step, the simulated data were compared to observed data. Rodatristat ethyl and rodatristat Tₘₐₓ, Cₘₐₓ, AUC (_{Auco-inf} or AUC₀₋₁₂ₕ) and t_{1/2 values}, simulated by the PBPK model were within 2-fold of observed data across the 11 clinical cohorts across the two studies used to verify the PBPK model in fed subjects. In most cases (91% and 85% for rodatristat ethyl and rodatristat, respectively), population simulated summary pharmacokinetic (PK) parameters (Tₘₐₓ, Cₘₐₓ and AUC) were within 1.5-fold of observed data.

The PBPK model was then applied to simulate drug-drug interactions (DDIs) with rodatristat ethyl and rodatristat acting as perpetrators of CYP- or transporter-mediated DDIs. PBPK simulations of each interaction scenario included 100 virtual subjects. Population estimates of geometric mean AUC_{0-inf} and Cₘₐₓ ratios were generated to predict the relative magnitude of each DDI.

The PBPK model was applied to simulate the interaction potential of multiple-dose RVT-1201 (600 mg BID). The simulated combined induction and competitive inhibition of CYP2B6 was not predicted to be clinically significant. The simulated bupropion Cₘₐₓ and AUC_{0-inf} geometric mean ratios under these conditions were 0.97 and 0.95, respectively. The simulated combined induction and competitive inhibition of CYP3A4 was categorized as ‘weak’ induction (≥1.25-fold; <2-fold change from baseline). Midazolam Cₘₐₓ and AUC_{0-inf} ratios under steady-state RVT-1201 conditions were 0.73 and 0.73, respectively (1.37-fold change from baseline).

No interaction was predicted in combination with single-dose rosiglitazone (CYP2C8 substrate), digoxin (P-gp substrate) or rosuvastatin (OATP1B1/3 substrate). The simulated Cₘₐₓ and AUC_{0-inf} ratios of these probe substrates under these conditions were <1.03 and 1.04, respectively.

### Introduction

RVT-1201 (AKA KAR5585, rodatristat ethyl) is a small molecule pro-drug that is rapidly hydrolyzed to KAR5417 (AKA KC0035, rodatristat), a potent tryptophan hydroxylate 1 (TPH1) inhibitor that reduces peripheral serotonin (5-hydroxytryptamine [5-HT]) levels in the gut mucosa, lung, and serum in animal models of pulmonary arterial hypertension. RVT-1201 is rapidly metabolized to form the active species KAR5417, which was the primary circulating species in a rat mass balance study (KRS-01), which is supported by human in vitro (XT184028) and clinical data (Clinical Study KAR5585-101, Clinical Study RVT-1201-1001).

RVT-1201 is a Biopharmaceutics Classification System (BCS) Class II or IV molecule with in vitro permeability consistent with oral bioavailability, which has been evaluated in a Phase 1 single ascending dose (SAD)/multiple ascending dose (MAD) study using a simple immediate release (IR) drug-in-capsule formulation (Clinical Study KAR5585-101). The active pharmaceutical ingredient used in this clinical study was polymorph Form 1. An optimized/enhanced oral formulation was developed with a goal to improve patient acceptance, increase the oral bioavailability relative to the IR capsule formulation to facilitate a dose reduction, reduce inter-subject variability and to optimize a formulation process suitable for larger scale manufacture. An oral IR tablet formulation and 2 spray-dried dispersion suspensions using polymorph Form 3 were investigated in a second clinical study (Clinical Study RVT-1201-1001). The purpose of this study was to assess the performance of selected formulations following oral administration to healthy subjects.

Clinical Study RVT-1201-1001 was a single and repeat dose study of RVT-1201 in healthy subjects to evaluate the pharmacokinetic profile of RVT-1201 and its active metabolite KAR5417. In Part 1 of this study, a single oral dose of 400 mg RVT-120 was administered as an oral powder in Regimen A and B, and as immediate release tablets in Regimen C, to 9 healthy male subjects. In addition, a single 1200 mg dose as immediate release tablets was administered in Regimen D.

Following oral administration in the fed state, RVT-1201 was well absorbed and peak plasma levels were achieved after a median time of 2.5 h. RVT-1201 plasma concentration was variable between subjects, and appeared to decline in an approximately mono-exponential manner (log scale), with an average geometric mean plasma terminal t_{1/2} of 5.2 h after 400 mg BID dosing (Clinical Studies RVT-1201-1001 and KAR5585-101). The planned clinical dose schedule of RVT-1201 in ELEVATE2 is up to 600 mg BID. The twice daily dose schedule is expected to result in minimal accumulation of RVT-1201 (<1.5-fold) after multiple dosing due to the elimination half-life of the drug. The geometric mean apparent clearance was 900 L/h after oral administration in Regimen C of Clinical Study RVT-1201-1001. Elimination of RVT-1201 was rapid, and primarily facilitated by hydrolysis to KAR5417. Formation of KAR5417 was attributed to de-esterification of RVT-1201.

KAR5417 is rapidly formed after oral administration of RVT-1201. The median Tₘₐₓ of KAR5417 in Regimen C of Clinical Study RVT-1201-1001 (400 mg single dose) was 3.50 h. The geometric mean Cₘₐₓ of KAR5417 (517 ng/mL) was approximately 5-fold greater than the Cₘₐₓ of RVT-1201. The geometric mean plasma terminal t_{1/2} of KAR5417 was 12.3 h, indicating that KAR5417 pharmacokinetics are elimination rate limited.

Data from one cohort (Regimen C, 400 mg single dose) of Clinical Study RVT-1201-1001 were used for model development and refinement. RVT-1201 and KAR5417 data from this cohort were used to develop the RVT-1201 absorption, and RVT-1201 and KAR5417 distribution and elimination components of the PBPK model.

Data from two clinical studies were used to verify the PBPK model (Clinical Study KAR5585-101 and Clinical Study RVT-1201-1001). Clinical Study KAR5585-101 was a randomized, double blind, placebo-controlled, Phase 1, first-in-human study to characterize the pharmacokinetics of single and multiple ascending doses of RVT-1201 in healthy subjects. RVT-1201 and KAR5417 plasma concentration data on Day 1, 7 and 14 of 400 mg BID administration in fed subjects were used to support verification of the PBPK model. In addition, multiple dose RVT-1201 and KAR5417 plasma concentration data on Day 1, 7 and 14 of 400 mg or 800 mg BID administration in fed subjects in Clinical Study RVT-1201-1001 were also used to support model verification.

In vitro inhibition studies indicated a potential for RVT-1201 and KAR5417 to inhibit specific CYP isoforms (CYP2B6, CYP2C8 (RVT-1201 only), CYP3A4) (XT155056) and drug transporters (P-gp (RVT-1201 only), OATP1B1/3) (XT-158039). mRNA induction of CYP isoforms (CYP2B6, CYP3A4) by RVT-1201 and KAR5417 was also observed in vitro (XT153040). Therefore, development and verification of a PBPK model for KAR5585 and KAR5417 was performed to support investigation of DDI risk in vivo.

The goal of this analysis was to investigate the clinical DDI potential of RVT-1201 (600 mg BID), as a perpetrator with substrates of CYP2B6, CYP2C8, CYP3A4, P-gp and OATP1B1/3.

### Modelling Strategy

A PBPK model was developed to predict in vivo human RVT-1201 and KAR5417 PK, based on available or simulator-predicted physicochemical, in vitro and clinical data. Model development, verification and application are summarized in the following.

### Model Development

Model development was completed using RVT-1201 and KAR5417 plasma concentration data after a 400 mg single dose of RVT-1201 as an immediate release tablet in Regimen C of Clinical Study RVT-1201-1001. The following steps were applied in developing the PBPK model.
Physicochemical and blood binding data (Molecular Weight (MW), Log P, pKa, fuₚ, blood to plasma ratio (B/P) were incorporated in the model based on experimental data
Oral absorption of RVT-1201 was described by a first order model
RVT-1201 and KAR5417 systemic distribution were described by a minimal PBPK model
Initial estimation of RVT-1201 volume of distribution parameters was based on in vitro Log P, pKa, fuₚ, B/P parameters and geometric mean CL_{PO} from Regimen C of Study RVT-1201-1001 (dataset used for model development)
RVT-1201 T_{lag} and kₐ were estimated based on the mean plasma concentration data from Regimen C of Clinical Study RVT-1201-1001
The Simulator estimate of RVT-1201 CLₒᵤₜ was optimized to approximate overall mean t_{1/2} of RVT-1201
RVT-1201 clearance, and KAR5417 formation were assigned to a cytosolic enzyme mediated process (initial estimate of RVT-1201 CLᵢₙₜ was based on liver S9 data and scaled up 6.5-fold to result in sufficient KAR5417 formation)
RVT-1201 volume of distribution parameters were re-estimated after change from CL_{PO} to cytosolic CIᵢₙₜ
KAR5417 volume of distribution parameters were initially set equal to those for RVT-1201; further refinement of deep distribution compartment volume was required to reproduce the observed PK profile from Regimen C of RVT-1201-Study RVT-1201-1001
KAR5417 CLPO was optimized to reproduce the observed PK profile from Regimen C of Study RVT-1201-1001
RVT-1201 and KAR5417 IC50 data were used to estimate Ki values
RVT-1201 and KAR5417 induction of CYP2B6 and CYP3A4 was incorporated in the model based on the in vitro induction of mRNA in human hepatocytes in incubations containing 0.1, 0.3, 1, 3, 10 or 20 μM RVT-1201 or KAR5417
Rifampin induction data were used to scale estimates of RVT-1201 and KAR5417 CYP3A4 Indₘₐₓ

### Model Verification

The PBPK model was verified by comparing population simulated plasma concentration-time profiles and summary PK parameters to observed data from the following clinical studies:
Study RVT-1201-1001 Regimen E; 400 mg IR BID (fed); Day 1
Study RVT-1201-1001 Regimen E; 400 mg IR BID (fed); Day 7
Study RVT-1201-1001 Regimen E; 400 mg IR BID (fed); Day 14
Study RVT-1201-1001 Regimen F; 800 mg IR BID (fed); Day 1
Study RVT-1201-1001 Regimen F; 800 mg IR BID (fed); Day 7
Study RVT-1201-1001 Regimen F; 800 mg IR BID (fed); Day 14
Study KAR5585-101; 400 mg BID (fed); Day 1 AM
Study KAR5585-101; 400 mg BID (fed); Day 1 PM
Study KAR5585-101; 400 mg BID (fed); Day 7 AM
Study KAR5585-101; 400 mg BID (fed); Day 7 PM
Study KAR5585-101; 400 mg BID (fed); Day 14 AM

### Model Application

The PBPK model was applied to simulate the DDI potential of RVT-1201 and KAR5417 (after steady-state dosing of RVT-1201 600 mg BID) for the following DDIs as a perpetrator. Simulated DDI magnitude was summarized by population simulated geometric mean Cₘₐₓ and AUC_{0-inf} ratios of the probe substrate.
As a perpetrator of CYP2B6 inhibition (bupropion)
As a perpetrator of CYP2C8 inhibition (rosiglitazone)
As a perpetrator of CYP3A4 inhibition (midazolam)
As a perpetrator of P-gp inhibition (digoxin)
As a perpetrator of OATP1B1/3 inhibition (rosuvastatin)

### Prediction of RVT-1201 and KAR5417 Clearance

The metabolic stability of RVT-1201 was measured in vitro in hepatocytes, liver microsomes, lung microsomes, intestinal S9, lung S9, liver S9 and whole blood. The major metabolite detected in incubations with human hepatocytes was KAR5417 (KRS-02). RVT-1201 was rapidly metabolized to form KAR5417, with <5% remaining after a 2 h incubation. In heat deactivated rat hepatocytes, <5% RVT-1201 remained after 2 h incubation in the same study. Therefore, RVT-1201 metabolism (and KAR5417 formation) are not dependent on oxidative or conjugative enzymatic processes. Formation of KAR5417 from RVT-1201 is a de-esterification process, which implicates an esterase or esterases as the major mechanism. The enzyme(s) responsible for RVT-1201 metabolism to form KAR5417 has not been identified but are considered to be carboxylesterase(s). In addition, RVT-1201 metabolism and KAR5417 formation were investigated in rat and human whole blood(XT184028). Complete loss of RVT-1201 was observed in rat blood after incubation of 2 μM RVT-1201 for 15 min. Complete conversion to KAR5417 was observed in these samples.

However, in human whole blood incubations, no loss of RVT-1201 was observed. This indicates that the de-esterification of RVT-1201 to form KAR5417 is not mediated by esterase activity in the blood in human. Conversion of RVT-1201 to KAR5417 was observed in human S9 fractions for lung, intestine and liver, indicating a cytosolic enzyme is responsible for metabolism of RVT-1201 and formation of KAR5417. Therefore, metabolism of RVT-1201 was assigned to a cytosolic carboxylesterase (CES1) within the PBPK model. Although the enzyme responsible for RVT-1201 elimination and KAR5417 formation has not been identified, CES1 was selected to represent the enzymatic metabolism of RVT-1201 for this fit-for-purpose PBPK model.

The intrinsic clearance of RVT-1210 in liver S9 fractions (XT184028) was 20 μL/min/mg protein.

Incorporation of this value in the PBPK model resulted in an under prediction of the geometric mean oral clearance of RCT-1201 observed in Regimen C of Clinical Study RVT-1201-1001. Therefore, additional liver S9 clearance was manually optimized to estimate the observed oral clearance of RVT-1201. In addition, the liver S9 intrinsic clearance estimate did not result in adequate levels of KAR5417 formation. Therefore, the liver S9 intrinsic clearance was increased 6.5-fold to 130 μL/min/mg, consistent with the scaling recommended by Wood et al. (2017) for human hepatocyte or human liver microsome data. Additional liver S9 clearance, leading to formation of other metabolites, was manually optimized to an estimate of 5000 μL/min/mg.

Clearance of KAR5417 within the PBPK model was manually optimized using the observed mean plasma concentration data of KAR5417 in Regimen C of Clinical Study RVT-1201-1001 (to a value of 60 L/h).

### Prediction of RVT-1201 and KAR5417 Interaction Potential

The potential of RVT-1201 and KAR5417 to perpetrate DDIs was investigated in vitro (XT158039, XT155056, XT153040).

The competitive inhibition of specific transporters was investigated for RVT-1201 and KAR5417 in transporter-expressing in vitro systems (XT158039). Inhibition of P-gp was evaluated using a Caco-2 cell line with digoxin as probe substrate. Inhibition of OATP1B1 and OATP1B3 was evaluated using a HEK293 cell line expressing these transporters with estradiol-17β-glucuronide as probe substrate. In addition, inhibition of other transporters was also investigated (BCRP, OAT1, OAT3, OCT2, MATE1, MATE2-K). The IC50 estimates for P-gp (RVT-1201) and OATP1B1/3 (RVT-1201 and KAR5417) supported further evaluation of inhibition DDI potential using the PBPK model. For each IC50 estimate, substrate concentration and Km were used to estimate RVT-1201 and KAR5417 Ki with Equation 1 (Cheng and Prusoff, 1973).

Ki=I50/(1+S/Km) Equation (1)

The competitive inhibition of specific CYP isoforms was investigated for RVT-1201 and KAR5417 in human liver microsomes (HLM) (XT155056). Of the 8 CYP isoforms evaluated in vitro (CYP1A2, CYP2B6, CYP2C8, CYP2C9, CYP2D6, CYP3A4, CYP3A5), RVT-1201 IC50 estimates were determined for CYP2B6, CYP2C8 and CYP3A4 only. KAR5417 competitively inhibited CYP2C8 only. For each IC50 estimate, substrate concentration and Km were used to estimate RVT-1201 and KAR5417 Ki.

The induction of specific CYP isoforms (CYP1A2, CYP2B6, CYP3A4) was investigated for RVT-1201 and KAR5417 in human hepatocytes (HH) (XT153040). RVT-1201 and KAR5417 induction of CYP2B6 and CYP3A4 mRNA was observed in these in vitro experiments. CYP2B6 and CYP3A4 mRNA induction by RVT-1201 and KAR5417 (Emax and EC50) were used to estimate the PBPK model parameters Indₘₐₓ and Ind_{C50}. Within the PBPK model, Ind_{C50} is equal to EC₅₀, the concentration of inducer that supports half maximal induction of the CYP isoform (estimated by a sigmoid three parameter fit to in vitro concentration-dependent mRNA induction data). Within the PBPK model, Indₘₐₓ represents the maximum fold induction relative to incubation with vehicle only (vehicle=1). The reported Eₘₐₓ for RVT-1201 and KAR5417 was estimated by sigmoid three parameter fit to in vitro concentration-dependent mRNA induction data. Baseline activity was set equal to zero, rather than one, therefore reported Eₘₐₓ was increased by adding one to each value to estimate Indₘₐₓ. CYP2B6 induction data was used in the PBPK model as reported. CYP3A4 induction data was scaled based on the maximum induction by rifampin (at 20 μM). Scaling of CYP3A4 induction using rifampin data from the same system leverages the extensive literature on rifampin induction in vitro and in vivo and allows adjustment of in vitro induction data based on the relative performance of rifampin in each in vitro incubation, and the empirical relationship between rifampin induction of CYP3A4 in vitro and in vivo.

### Prediction of Nonspecific Protein Binding in Microsomes and Hepatocytes

Microsomal protein binding (fumic) is used to adjust for the impact of protein binding on intrinsic clearance and inhibition potency in in vivo experiments. Fumic values can be measured in vivo or predicted within the Simcyp Simulator. This relationship is summarized in Equation (2), where [P]mic is the microsomal protein concentration (mg/mL) used in the in vitro incubation.

fumic=1/[([P]mic×10^{0.646+log p-2.236})+1] Equation (2)

Hepatocyte protein binding (fuinc) is used to adjust for the impact of protein binding in human hepatocyte incubations. Fuinc may be predicted according to the methodology proposed by Kilford, et al. (2008) based on lipophilicity and estimated volume ratio in human hepatocytes. This relationship is summarized in Equation (3), where VR=0.005 at hepatocyte concentration of 106 cells/mL.

fuₕₑₚ=1/(1+125·V_{R}·10^{0.072-log(p/D2)+0.067-log(P/D)-1.126}) Equation (3)

Protein binding in HLM and HH incubations to evaluate the competitive inhibition and induction potential of RVT-1201 and KAR5417 for CYP isoforms was not reported. Therefore, free fraction in the in vitro inhibition and induction experiments was predicted.

### PBPK Model Application

The PBPK model was then applied to predict the impact of RVT-1201 and KAR5417, as inducers (RVT-1201 and KAR5417) and competitive inhibitor (RVT-1201), on the PK of sensitive substrates of CYP2B6 (bupropion) and CYP3A4 (midazolam), the impact of KAR5417 competitive inhibition on the PK of sensitive substrates of CYP2C8 (rosiglitazone), the impact of RVT-1201 on the PK of sensitive substrates of P-gp (digoxin) and the impact of RVT-1201 and KAR5417 on the PK of sensitive substrates of OATP1B1/3 (rosuvastatin). Simulations were completed using the simulated fed Healthy Volunteer population; age 18-55 years, 50% female. All PBPK simulations were conducted for 14 days of 400 mg BID RVT-1201, with a single-dose of probe substrate administered on Day 14. Each DDI simulation was composed of 10 virtual trials of 10 virtual subjects each. For each simulation, population summary PK parameters (based on a total n=100) were summarized.

### Discussion

A PBPK model was developed, which accurately simulated the observed plasma concentration of RVT-1201 and KAR5417 after single and multiple oral doses of RVT-1201 (400 mg BID, 800 mg BID). RVT-1201 and KAR5417 are weak concentration-dependent inducers of CYP2B6 and CYPP3A4 mRNA in vitro. The PBPK model was applied to simulate DDI potential when RVT-1201, dosed to steady state (600 mg BID), is co-administered with a single dose of a sensitive probe substrate of CYP2B6 or CYP3A4. In addition, RVT-1201 and KAR5417 were weak competitive inhibitors of CYP2B6, CYP2C8, CYP3A4, P-gp (RVT-1201 only) and OATP1B1/3 in vitro. The PBPK model was also applied to simulate the DDI potential when RVT-1201, dosed to steady state (600 mg BID), is co-administered with a single dose of a sensitive probe substrate of CYP2B6, CYP2C8, CYP3A4, P-gp or OATP1B1/3.

The PBPK model was developed using physicochemical, in vitro and clinical data collected for RVT-1201 and KAR5417. Single dose (400 mg) plasma concentration data (Regimen C of Clinical Study RVT-1201-1001) was used to estimate the distribution parameters of RVT-1210 and KAR5417. In addition, RVT-1201 and KAR5417 plasma concentration data from this dataset were used to refine estimates of enzymatic clearance of RVT-1201 and oral clearance of KAR5417.

The PBPK model was verified through application of the model to simulate RVT-1201 and KAR5417 plasma concentration data after a 400 mg BID or 800 mg BID for 14 days (Day 1, 7 and 14) in Clinical Study RVT-1201-1001 (Regimen E and F, respectively). In addition, 14 day administration of 400 mg BID in Clinical Study KAR5585-101 (Day 1 (AM and PM), 7 (AM and PM) and 14) was used for model verification.

Simulated population summary RVT-1201 Tₘₐₓ, Cₘₐₓ and AUC (AUC_{0-inf} after single dose; AUC₀₋₁₂ₕ after multiple doses) were within 0.40-1.60-fold, 0.76-1.38-fold and 0.71-1.58-fold, respectively, of observed RVT-1201 data (the majority (30 of 33 estimates) of simulated summary PK parameters were within 1.5-fold of observed). Based on the performance of the model in simulating these 11 datasets (33 simulated summary PK parameters), the RVT-1201 PBPK model is adequate for the simulation of DDI potential as perpetrator of DDIs.

Simulated population summary KAR5417 Tₘₐₓ, Cₘₐₓ and AUC (AUC_{0-inf} after single dose; AUC₀₋₁₂ₕ after multiple doses) were within 0.52-1.30-fold, 0.60-1.00-fold and 0.74-1.17-fold, respectively, of observed RVT-1201 data (the majority (28 of 33 estimates) of simulated summary PK parameters were within 1.5-fold of observed). Based on the performance of the model in simulating these 11 datasets (33 simulated summary PK parameters), the RVT-1201 PBPK model is adequate for the simulation of DDI potential as perpetrator of DDIs.

The PBPK model was applied to simulate the DDI potential of RVT-1201 and its metabolite KAR5417 as a perpetrator of CYP2B6, CYP2C8, CYP3A4, P-gp and OATP1B1/3-mediated DDIs.

The simulated DDI potential of RVT-1201 as an inducer of CYP2B6 was below the lower threshold of ‘weak’ (≥1.25-fold; <2-fold change from baseline, or exposure ratios of >0.50; ≤0.80), based on the estimate of CYP2B6 induction potential (in vitro induction of CYP2B6 mRNA). Under steady-state conditions for RVT-1201 and KAR5417, no DDI is predicted with sensitive substrates of CYP2B6. Induction of CYP mRNA is considered a more sensitive marker of induction than in vitro activity (Fahmi et al., 2010). Therefore, induction simulations based on mRNA data may be considered the ‘worst-case’ scenario of CYP2B6-mediated DDI liability of RVT-1201, and as such, are expected to represent an over-prediction of the clinical induction potential of RVT-1201 and KAR5417. The simulated DDI potential of RVT-1201 as an inducer of CYP3A4 was at the lower threshold of ‘weak’ (≥1.25-fold; <2-fold), based on the estimate of CYP3A4 induction potential (in vitro induction of CYP3A4 mRNA). Under steady-state conditions for RVT-1201 and KAR5417, a weak DDI is predicted with sensitive substrates of CYP3A4. Induction of CYP mRNA is considered a more sensitive marker of induction than in vitro activity (Fahmi et al., 2010). Therefore, induction simulations based on mRNA data may be considered the ‘worst-case’ scenario of CYP3A4-mediated DDI liability of RVT-1201, and as such, are expected to represent an over-prediction of the clinical induction potential of RVT-1201 and KAR5417. However, for DDI simulations, the PBPK model incorporated a rifampin-scaled estimate of Indmax which is expected to minimize the over-prediction associated with mRNA-based induction parameters.

The simulated DDI potential of RVT-1201 as a competitive inhibitor of CYP2C8 was below the lower threshold of ‘weak’ (≥1.25-fold; <2-fold), based on the estimate of CYP2C8 inhibition potential (in vitro Ki). Under steady-state conditions for RVT-1201 and KAR5417, no DDI is predicted with sensitive substrates of CYP2C8. Under ‘worst-case’ scenario conditions where the in vitro Ki of RVT-1201 and KAR5417 were reduced 10-fold, no DDI with sensitive substrates of CYP2C8 is predicted.

The simulated DDI potential of RVT-1201 as a competitive inhibitor of P-gp was below the lower threshold of ‘weak’ (≥1.25-fold; <2-fold), based on the estimate of P-gp inhibition potential (invitro Ki). Under steady-state conditions for RVT-1201 and KAR5417, no DDI is predicted with sensitive substrates of P-gp. Under ‘worst-case’ scenario conditions where the in vitro Ki of RVT-1201 and KAR5417 were reduced 15-fold, no DDI with sensitive substrates of P-gp is predicted.

The simulated DDI potential of RVT-1201 as a competitive inhibitor of OATP1B1/3 was below the lower threshold of ‘weak’ (≥1.25-fold; <2-fold), based on the estimate of OATP1B1/3 inhibition potential (in vitro Ki). Under steady-state conditions for RVT-1201 and KAR5417, no DDI is predicted with sensitive substrates of OATP1B1/3. Under ‘worst-case’ scenario conditions where the in vitro Ki of RVT-1201 and KAR5417 were reduced 10-fold, no DDI with sensitive substrates of OATP1B1/3 is predicted.

In conclusion, a PBPK model was developed which described the observed plasma concentration data of RVT-1201 and KAR5417 after oral administration of single or multiple (BID) doses between 400 and 800 mg. This model was then applied to simulate the DDI potential of RVT-1201 and its metabolite KAR5417, as perpetrator of CYP2B6, CYP2C8, CYP3A4, P-gp or OATP1B1/3 DDIs. A ‘weak’ potential for CYP3A4-mediated DDIs was the only interaction predicted by the PBPK model. RVT-1201 is not predicted to perpetrate DDIs with sensitive substrates of CYP2B6, CYP2C8, P-gp or OATP1B1/3.

### Abbreviations

ADME drug absorption, distribution, metabolism, excretion
AUC_{0-inf} area under the curve extrapolated to infinity

### BCS Biopharmaceutics Classification System

BID twice a day
B/P blood to plasma ratio
CI confidence interval
CLᵢₙ input rate clearance (VSAC)
C_{Loral} oral clearance
CLₒᵤₜ elimination rate clearance (VSAC)
CLₚₑᵣₘ a clearance term defining the permeability through the enterocyte
CL_{PO} oral plasma clearance
CLᵣₑₙₐₗ renal clearance
CL_{u,int,g} intrinsic clearance of the unbound drug in the gut
CL_{u,int,H} unbound hepatic intrinsic clearance
Cₘₐₓ maximal drug concentration
CYP cytochrome P450 enzyme
DDI drug-drug interaction
EC₅₀ effective concentration of an agonist that leads to 50% of the maximal possible effect of that agonist
Eₘₐₓ Maximum induction
E_{G} fraction undergoing first pass metabolism in the gut
E_{H} fraction undergoing first pass metabolism in the liver
fₐ fraction absorbed from the gut
F_{G} fraction escaping first pass metabolism in the gut
fm_{CYP} fraction metabolized by individual CYP isoforms
fu_{b} fraction unbound in blood
fu_{gut} fraction unbound in the gut
fu_{inc} fraction unbound in hepatocyte
fu_{mic} fraction unbound in an in-vitro microsomal preparation
fuₚ fraction unbound in plasma
h hours
HH human hepatocyte
HIM human intestine microsome
HLM human liver microsome
IC₅₀ half maximal inhibitory concentration
Indₘₐₓ maximal fold induction
IR immediate release
ISEF inter-system extrapolation factor
kₐ first order absorption rate constant
kg kilograms
Kᵢ enzyme/transporter competitive inhibition constant (concentration of inhibitor associated with half maximal inhibition)
Log P Log of the octanol-water partition coefficient for the neutral compound
μ micro-
M molar
MAD multiple ascending dose
Mg milligram
MW molecular weight
Ng nanogram
Pₐₚₚ apparent permeability
PBPK physiologically based pharmacokinetic
P_{eff} effective intestinal membrane permeability
P_{eff,man} effective human jejunum permeability
PK pharmacokinetic
pKa acid dissociation constant
QD once a day
Q_{gut} flow rate for overall delivery of drug to the gut
Q_{H} blood flow in the liver
Q_{HA} blood flow in the hepatic artery
Q_{PV} blood flow in the portal vein
Qᵥᵢₗₗᵢ villous blood flow
rhCYP recombinant human CYP

### S/O Simulated/Observed

SAC single adjusting compartment
SAD single ascending dose
t_{1/2} half-life
TIW three times a week
T_{lag} lag time
Tₘₐₓ time at which maximum plasma concentration of drug is reached
V18 version 18
Vᵣ ratio of the cell and incubation volume
V_{SAC} volume of single adjusting compartment
V_{SS} volume of distribution at steady state
WLS weighted least square

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances which fall within the scope of the appended claims.

1.-88. (canceled)

89. A method for treating pulmonary arterial hypertension, comprising administering daily to a human patient in need thereof (A) an amount of up to 1200 mg of a first compound of rodatristat ethyl and (B) an amount of a second compound selected from the group consisting of ambrisentan, sildenafil, tadalafil, bosentan, treprostinil, selexipag, macitentan, and two or more of the foregoing,
wherein the first compound and the second compound are in a combination of that results in an additive or synergistic reduction in symptoms of pulmonary arterial hypertension compared to the first compound and the second compound alone with a low risk for drug-drug interactions.

90. The method of claim 89, wherein the rodatristat ethyl is rodatristat ethyl, Form 3.

91. The method of claim 89, wherein the rodatristat ethyl is rodatristat ethyl, Form 1.

92. The method of claim 89, wherein the rodatristat ethyl is rodatristat ethyl, amorphous.

93. A method for treating pulmonary arterial hypertension, comprising administering to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing,
wherein the first compound and the second compound are in a combination that results in a synergistic or additive reduction in symptoms of pulmonary arterial hypertension compared to the first compound and the second compound alone, and
wherein the amount of rodatristat ethyl administered results in a C_{max, unbound} for rodatristat ethyl and its active metabolite rodatristat in the bloodstream of less than 0.028 μM.

94. The method of claim 93, wherein the rodatristat ethyl is rodatristat ethyl, Form 3.

95. The method of claim 93, wherein the rodatristat ethyl is rodatristat ethyl, Form 1.

96. The method of claim 93, wherein the rodatristat ethyl is rodatristat ethyl, amorphous.

97. A method for treating pulmonary arterial hypertension, comprising administering to a human patient in need thereof (A) an amount of a first compound of rodatristat ethyl and (B) an amount of a second compound selected from the group consisting of ambrisentan, sildenafil, tadalafil, bosentan, treprostinil, selexipag, macitentan, and two or more of the foregoing,
wherein the first compound and the second compound are in a combination that results in a synergistic reduction in side effects of pulmonary arterial hypertension compared to the first compound and the second compound alone or additively, and
wherein the amount of rodatristat ethyl administered results in a C_{max, unbound} for rodatristat ethyl and its active metabolite rodatristat in the bloodstream of less than 0.028 μM.

98. The method of claim 97, wherein the rodatristat ethyl is rodatristat ethyl, Form 3.

99. The method of claim 97, wherein the rodatristat ethyl is rodatristat ethyl, Form 1.

100. The method of claim 97, wherein the rodatristat ethyl is rodatristat ethyl, amorphous.

101. A method for treating pulmonary arterial hypertension, comprising administering daily to a human patient in need thereof (A) an amount of a first compound of rodatristat ethyl, and (B) an amount of a second compound selected from the group consisting of ambrisentan, sildenafil, tadalafil, bosentan, treprostinil, selexipag, macitentan, and any combination thereof,
wherein the first compound and the second compound are in a combination that results in an additive or synergistic reduction in side effects of pulmonary arterial hypertension compared to the first compound and the second compound alone, and
wherein neither the first compound nor its active metabolite rodatristat substantially impact metabolism of the second compound in the bloodstream.

102. The method of claim 101, wherein the first compound is rodatristat ethyl, Form 3.

103. The method of claim 101, wherein the rodatristat ethyl is rodatristat ethyl, Form 1.

104. The method of claim 101, wherein the rodatristat ethyl is rodatristat ethyl, amorphous.

105. A method for treating pulmonary arterial hypertension, comprising administering daily to a human patient in need thereof (A) an amount of a first compound of rodatristat ethyl, and (B) an amount of a second compound selected from the group consisting of ambrisentan, sildenafil, tadalafil, bosentan, treprostinil, selexipag, macitentan, and any combination thereof,
wherein the first compound exhibits an IC₅₀ of ≥30 μM for one or more of the enzyme group consisting of CYP1A2, CYP2C9, CYP2C19, and CYP2D6 in the bloodstream.

106. The method of claim 105, wherein the first compound is rodatristat ethyl, Form 3.

107. The method of claim 105, wherein the rodatristat ethyl is rodatristat ethyl, Form 1.

108. The method of claim 105, wherein the rodatristat ethyl is rodatristat ethyl, amorphous.

109. A method for treating pulmonary arterial hypertension, comprising administering daily to a human patient in need thereof: (A) an amount of a first compound of rodatristat ethyl, and (B) an amount of a second compound selected from the group consisting of ambrisentan, sildenafil, tadalafil, bosentan, treprostinil, selexipag, macitentan, and any combination thereof,
wherein the active metabolite rodatristat of the first compound exhibits an IC₅₀ of ≥30 μM for one or more of the enzyme group consisting of CYP1A2, CYP2B6, CYP2C9, CYP2C19, CYP2D6, and CYP3A4.

110. The method of claim 109, wherein the first compound is rodatristat ethyl, Form 3.

111. The method of claim 109, wherein the rodatristat ethyl is rodatristat ethyl, Form 1.

112. The method of claim 109, wherein the rodatristat ethyl is rodatristat ethyl, amorphous.
